SEQUENCE LISTING (43 sequences)

SEQ ID NO: 1 - DNA, 190 bp, Mus sp.
atgtctcgaggtgccgacgtggaaagaccgagagatcccgccaacgggtcatacccgttgacaagtaccaatctctactccagcgcaaccagtaggtaccgagatcggagatcaaaaagggtgtccgcgacgacgacgacgacgacaacgacgacgacgacgacgacgacgacgacgcacagaccctggg

SEQ ID NO: 2 - DNA, 59 bp, Mus sp.
cagccaagagcagagccaccaggacagagacaccggtgtacagagctccacggctgcac

SEQ ID NO: 3 - DNA, 144 bp, Mus sp.
cagttcacgccatccatggagttcttaagatgcttcagattctctgggaaggtccccttcagctgcgggtactccaggggtccagacctcgtgagcagatgcatcacatggtcctgggtcatgttgccgtacttggtaacgttc

SEQ ID NO: 4 - DNA, 207 bp, Homo sapiens
gtggcctggccagcgaggagcagagtcaccaggatggaaaagcctgtgtacagggctccgcggctgcacttgctctccggggccccagggcgccggcccagcatgggcagttgctcattgttggagataaggtcgcgctggtcatccatgactggcttctgatcttcccgacagctcctgcttctcctcctgtgcatctgggaccct

SEQ ID NO: 5 - DNA, 326 bp, Homo sapiens
tgctcacaggcttgggaggcttgggaagcttcatgcgcaggttctccagctgcaggttctgggaggtgactgtcagtttgtccagccggccctgctgctggtacaggaagtaggcggtggtggcctggccagcgaggagcagagtcaccaggatggaaaagcctgtgtacagggctccgcggctgcacttgctctccggggccccagggcgccggcccagcatgggcagttgctcattgttggagataaggtcgcgctggtcatccatgactggcttctgatcttcccgacagctcctgcttctcctcctgtgcatctgggaccct

SEQ ID NO: 6 - DNA, 436 bp, Homo sapiens
cacatggtcctctgtcatgttgccatacttggtggcattctgcatgggcccctggggcagggctcccatgggcagcgcctgcatcagcagcggggtggccatgcgcatcttgctcacaggcttgggaggcttgggaagcttcatgcgcaggttctccagctgcaggttctgggaggtgactgtcagtttgtccagccggccctgctgctggtacaggaagtaggcggtggtggcctggccagcgaggagcagagtcaccaggatggaaaagcctgtgtacagggctccgcggctgcacttgctctccggggccccagggcgccggcccagcatgggcagttgctcattgttggagataaggtcgcgctggtcatccatgactggcttctgatcttcccgacagctcctgcttctcctcctgtgcatctgggaccct

SEQ ID NO: 7 - DNA, 493 bp, Homo sapiens
gctccccttcagtggcgggtacaccttcagggggtcagcattctggagcaggtgcatcacatggtcctctgtcatgttgccatacttggtggcattctgcatgggcccctggggcagggctcccatgggcagcgcctgcatcagcagcggggtggccatgcgcatcttgctcacaggcttgggaggcttgggaagcttcatgcgcaggttctccagctgcaggttctgggaggtgactgtcagtttgtccagccggccctgctgctggtacaggaagtaggcggtggtggcctggccagcgaggagcagagtcaccaggatggaaaagcctgtgtacagggctccgcggctgcacttgctctccggggccccagggcgccggcccagcatgggcagttgctcattgttggagataaggtcgcgctggtcatccatgactggcttctgatcttcccgacagctcctgcttctcctcctgtgcatctgggaccct

SEQ ID NO: 8 - DNA, 574 bp, Homo sapiens
ccaatggtgcatccagctctcaaagaccttccagtctatggtctccatggtgttcttaaggtgtctcaggttctccgggaagctccccttcagtggcgggtacaccttcagggggtcagcattctggagcaggtgcatcacatggtcctctgtcatgttgccatacttggtggcattctgcatgggcccctggggcagggctcccatgggcagcgcctgcatcagcagcggggtggccatgcgcatcttgctcacaggcttgggaggcttgggaagcttcatgcgcaggttctccagctgcaggttctgggaggtgactgtcagtttgtccagccggccctgctgctggtacaggaagtaggcggtggtggcctggccagcgaggagcagagtcaccaggatggaaaagcctgtgtacagggctccgcggctgcacttgctctccggggccccagggcgccggcccagcatgggcagttgctcattgttggagataaggtcgcgctggtcatccatgactggcttctgatcttcccgacagctcctgcttctcctcctgtgcatctgggaccct

SEQ ID NO: 9 - DNA, 700 bp, Homo sapiens
gactgggcccagatcctgcttggtcacacccagcccagaagacgggtcctccagttccagtgactctttcggtggagcgtcagtgggcttttgctccaaggagtgcctgctcatttcaaacaggagccaatggtgcatccagctctcaaagaccttccagtctatggtctccatggtgttcttaaggtgtctcaggttctccgggaagctccccttcagtggcgggtacaccttcagggggtcagcattctggagcaggtgcatcacatggtcctctgtcatgttgccatacttggtggcattctgcatgggcccctggggcagggctcccatgggcagcgcctgcatcagcagcggggtggccatgcgcatcttgctcacaggcttgggaggcttgggaagcttcatgcgcaggttctccagctgcaggttctgggaggtgactgtcagtttgtccagccggccctgctgctggtacaggaagtaggcggtggtggcctggccagcgaggagcagagtcaccaggatggaaaagcctgtgtacagggctccgcggctgcacttgctctccggggccccagggcgccggcccagcatgggcagttgctcattgttggagataaggtcgcgctggtcatccatgactggcttctgatcttcccgacagctcctgcttctcctcctgtgcatctgggaccct

SEQ ID NO: 10 - DNA, 556 bp, Homo sapiens
ctgcagggggggggggggggggggggggggagggatgggtggaaaacattggctcttccttggggagtgatgctggggaaagggaagagagtggctcagcctgcaggtaaataggctagaaaagccaaggccaaaggctggaggggagaggacagtcagcatgtccagcctggggtctgggtgtagggttatcccttctccctgtgccttcccatctcgtccatgagcctaggtcttggagccttgtgttggaggctgctgtgatgtcaggaacggggatctgtctagcttttggccacttcctgggacctcacgcccctgttgacagatggagattgggcagcagggccttgctgcattgttatctgctgttccgacttggtttgtcttgtccaagggtgacgaaagagccaggcaccagggtctcatgggatgaggtacagggtgggagatgggggaggggctgggggctgaagggagcaagaaagctgtagctgtgtggggctggcaggatgttgaagaccgcctctgctgctctcacatggggactgggccc

SEQ ID NO: 11 - DNA, 57 bp, Artificial Sequence
  Description of Artificial Sequence Synthetic SiRNA construct
tcgattcccagatgcacaggaggagatcgatctcctcctgtgcatctgggaattttt

SEQ ID NO: 12 - DNA, 57 bp, Artificial Sequence
  Description of Artificial Sequence Synthetic SiRNA construct
tcgaatgcacaggaggagaagcaggatcgatcctgcttctcctcctgtgcatttttt

SEQ ID NO: 13 - DNA, 57 bp, Artificial Sequence
  Description of Artificial Sequence Synthetic SiRNA construct
tcgaaagccagtcatggatgaccagatcgatctggtcatccatgactggcttttttt

SEQ ID NO: 14 - DNA, 57 bp, Artificial Sequence
  Description of Artificial Sequence Synthetic SiRNA construct
tcgaatggatgaccagcgcgaccttatcgataaggtcgcgctggtcatccatttttt

SEQ ID NO: 15 - DNA, 57 bp, Artificial Sequence
  Description of Artificial Sequence Synthetic SiRNA construct
tcgacaatgagcaactgcccatgctatcgatagcatgggcagttgctcattgttttt

SEQ ID NO: 16 - DNA, 57 bp, Artificial Sequence
  Description of Artificial Sequence Synthetic SiRNA construct
tcgacctgcagctggagaacctgcgatcgatcgcaggttctccagctgcaggttttt

SEQ ID NO: 17 - DNA, 57 bp, Artificial Sequence
  Description of Artificial Sequence Synthetic SiRNA construct
tcgagcctgtgagcaagatgcgcatatcgatatgcgcatcttgctcacaggcttttt

SEQ ID NO: 18 - DNA, 57 bp, Artificial Sequence
  Description of Artificial Sequence Synthetic SiRNA construct
tcgatgccaccaagtatggcaacatatcgatatgttgccatacttggtggcattttt

SEQ ID NO: 19 - DNA, 57 bp, Artificial Sequence
  Description of Artificial Sequence Synthetic SiRNA construct
tcgacatgacagaggaccatgtgatatcgatatcacatggtcctctgtcatgttttt

SEQ ID NO: 20 - DNA, 57 bp, Artificial Sequence
  Description of Artificial Sequence Synthetic SiRNA construct
tcgacctgagacaccttaagaacacatcgatgtgttcttaaggtgtctcaggttttt

SEQ ID NO: 21 - RNA, 21 nt, Artificial Sequence
  Description of Artificial Sequence Synthetic SiRNA construct
ccauuggcuccuguuugaauu

SEQ ID NO: 22 - RNA, 21 nt, Artificial Sequence
  Description of Artificial Sequence Synthetic SiRNA construct
gugguaaccgaggacaaacuu

SEQ ID NO: 23 - RNA, 21 nt, Artificial Sequence
  Description of Artificial Sequence Synthetic SiRNA construct
cacugacgcuccaccgaaauu

SEQ ID NO: 24 - RNA, 21 nt, Artificial Sequence
  Description of Artificial Sequence Synthetic SiRNA construct
gggugacugcgagguggcuuu

SEQ ID NO: 25 - RNA, 21 nt, Artificial Sequence
  Description of Artificial Sequence Synthetic SiRNA construct
gaacuggaggacccgucuuuu

SEQ ID NO: 26 - RNA, 21 nt, Artificial Sequence
  Description of Artificial Sequence Synthetic SiRNA construct
accuugaccuccugggcagaa

SEQ ID NO: 27 - RNA, 21 nt, Artificial Sequence
  Description of Artificial Sequence Synthetic SiRNA construct
gggugugaccaagcaggauuu

SEQ ID NO: 28 - RNA, 21 nt, Artificial Sequence
  Description of Artificial Sequence Synthetic SiRNA construct
gacccacacugguucguccua

SEQ ID NO: 29 - RNA, 21 nt, Homo sapiens
uucccagaugcacaggaggag

SEQ ID NO: 30 - RNA, 21 nt, Homo sapiens
augcacaggaggagaagcagg

SEQ ID NO: 31 - RNA, 21 nt, Homo sapiens
aagccagucauggaugaccag

SEQ ID NO: 32 - RNA, 21 nt, Homo sapiens
auggaugaccagcgcgaccuu

SEQ ID NO: 33 - RNA, 21 nt, Homo sapiens
caaugagcaacugcccaugcu

SEQ ID NO: 34 - RNA, 21 nt, Homo sapiens
ccugcagcuggagaaccugcg

SEQ ID NO: 35 - RNA, 21 nt, Homo sapiens
gccugugagcaagaugcgcau

SEQ ID NO: 36 - RNA, 21 nt, Homo sapiens
ugccaccaaguauggcaacau

SEQ ID NO: 37 - RNA, 19 nt, Homo sapiens
ccauuggcuccuguuugaa

SEQ ID NO: 38 - RNA, 19 nt, Homo sapiens
cacugacgcuccaccgaaa

SEQ ID NO: 39 - RNA, 19 nt, Homo sapiens
gaacuggaggacccgucuu

SEQ ID NO: 40 - RNA, 19 nt, Homo sapiens
gggugugaccaagcaggau

SEQ ID NO: 41 - DNA, 15 bp, Artificial Sequence
  Description of Artificial Sequence Synthetic oligonucleotide
ggtcatccatggctc

SEQ ID NO: 42 - DNA, 1327 bp, Homo sapiens
cagggtcccagatgcacaggaggagaagcaggagctgtcgggaagatcagaagccagtcatggatgaccagcgcgaccttatctccaacaatgagcaactgcccatgctgggccggcgccctggggccccggagagcaagtgcagccgcggagccctgtacacaggcttttccatcctggtgactctgctcctcgctggccaggccaccaccgcctacttcctgtaccagcagcagggccggctggacaaactgacagtcacctcccagaacctgcagctggagaacctgcgcatgaagcttcccaagcctcccaagcctgtgagcaagatgcgcatggccaccccgctgctgatgcaggcgctgcccatgggagccctgccccaggggcccatgcagaatgccaccaagtatggcaacatgacagaggaccatgtgatgcacctgctccagaatgctgaccccctgaaggtgtacccgccactgaaggggagcttcccggagaacctgagacaccttaagaacaccatggagaccatagactggaaggtctttgagagctggatgcaccattggctcctgtttgaaatgagcaggcactccttggagcaaaagcccactgacgctccaccgaaagagtcactggaactggaggacccgtcttctgggctgggtgtgaccaagcaggatctgggcccagtccccatgtgagagcagcagaggcggtcttcaacatcctgccagccccacacagctacagctttcttgctcccttcagcccccagcccctcccccatctcccaccctgtacctcatcccatgagaccctggtgcctggctctttcgtcacccttggacaagacaaaccaagtcggaacagcagataacaatgcagcaaggccctgctgcccaatctccatctgtcaacaggggcgtgaggtcccaggaagtggccaaaagctagacagatccccgttcctgacatcacagcagcctccaacacaaggctccaagacctaggctcatggacgagatgggaaggcacagggagaagggataaccctacacccagaccccaggctggacatgctgactgtcctctcccctccagcctttggccttggcttttctagcctatttacctgcaggctgagccactctcttccctttccccagcatcactccccaaggaagagccaatgttttccacccataatcctttctgccgacccctagttccctctgctcagccaagcttgttatcagctttcagggccatggttcacattagaataaaaggtagtaattagaaaaaaaaaaaaaaaaaa

SEQ ID NO: 43 - protein, 15 aa, Artificial Sequence
  Description of Artificial Sequence Synthetic peptide
RIQRGPGRAFVTIGK

(19) United States
(12) Patent Application Publication
(10) Pub. No.: US 2006/0008448 A1
(43) Pub. Date: Jan. 12, 2006

(54) Inhibition of li expression in mammalian cells

(76) Inventors: Minzhen Xu, Northborough, MA (US); Robert Humphreys, Acton, MA (US)

Correspondence Address: Kevin M. Farrell;Pierce Atwood, Suite 350, One New Hampshire Avenue, Portsmouth, NH, 03801, US

(21) Appl. No.: 10/999,208

(22) Filed: Nov. 29, 2004

Related U.S. Application Data
(63) Continuation-in-part of application No. 10/127,347, filed on Apr. 22, 2002.
Application No. 10/127,347 is a continuation-in-part of application No. 10/054,387, filed on Jan. 22, 2002, now abandoned.
Application No. 10/054,387 is a division of application No. 09/205,995, filed on Dec. 4, 1998, now Pat. No. 6,368,855.
Application No. 09/205,995 is a continuation-in-part of application No. 09/036,746, filed on Mar. 9, 1998, now abandoned.
Application No. 09/036,746 is a continuation of application No. 08/661,627, filed on Jun. 11, 1996, now Pat. No. 5,726,020.

(51) Int. Cl. A61K 48/00

(52) U.S. Cl. 424/93.2

(57) ABSTRACT

The present invention is directed toward compositions and methods involving the inhibition of Ii expression in cells for the purpose of altering antigen presentation pathways. More specifically, disclosed are compositions and methods which relate to MHC Class II molecule presentation of antigenic epitopes which, under normal circumstances, would not be presented in association with MHC Class II molecules. The invention relates to presentation in cells which normally express MHC Class II molecules, as well as cells which can be induced to express MHC Class II molecules. Embodiments relating to RNA interference of Ii are specifically disclosed.

### RELATED APPLICATIONS

This application is a continuation-in-part of prior U.S. application Ser. No. 10/127,347, filed Apr. 22, 2002 and Ser. No. 10/054,387, filed Jan. 22, 2002, which is a division of Ser. No. 09/205,995, filed Dec. 4, 1998, now U.S. Pat. No. 6,368,855, issued Apr. 9, 2002, which is a continuation-in-part of Ser. No. 09/036,746, filed Mar. 9, 1998, now abandoned, which is a continuation of Ser. No. 08/661,627, filed Jun. 11, 1996, now U.S. Pat. No. 5,726,020, issued Mar. 10, 1998.

### BACKGROUND OF THE INVENTION

The immune response to specific antigens is regulated by the recognition of peptide fragments of those antigens by T lymphocytes. Within an antigen presenting cell, peptide fragments of the processed antigen become bound into the antigenic peptide binding site of major histocompatibility complex (MHC) molecules. These peptide-MHC complexes are then transported to the cell surface for recognition (of both the foreign peptide and the adjacent surface of the presenting MHC molecule) by T cell receptors on helper or cytotoxic T lymphocytes. There are two classes of MHC molecules that deliver peptides, MHC class I and MHC class II.

MHC class I molecules present antigen to CD8-positive cytotoxic T-lymphocytes, which then become activated and can kill the antigen presenting cell directly. Class I MHC molecules exclusively receive peptides from endogenously synthesized proteins, such as an infectious virus, in the endoplasmic reticulum at around the time of their synthesis.

MHC class II molecules present antigen to CD4-positive helper T-lymphocytes (T helper cells). Once activated, T helper cells contribute to the activation of cytotoxic T lymphocytes (T killer cells) and B lymphocytes via physical contact and cytokine release. Unlike MHC class I molecules, MHC class II molecules bind exogenous antigens which have been internalized via non-specific or specific endocytosis. Around the time of synthesis MHC class II molecules are blocked from binding endogenous antigen by instead binding the invariant chain protein (Ii). These MHC class II-Ii protein complexes are transported from the endoplasmic reticulum to a post-Golgi compartment where Ii is released by proteolysis and exogenous antigenic peptides are bound (Daibata et al., Molecular Immunology 31: 255-260 (1994); Xu et al., Molecular Immunology 31: 723-731 (1994)).

MHC class I and MHC class II molecules have a distinct distribution among cells. Almost all nucleated cells express MHC class I molecules, although the level of expression varies between cell types. Cells of the immune system express abundant MHC class I on their surfaces, while liver cells express relatively low levels. Non-nucleated cells express little or no MHC class I. MHC Class II molecules are highly expressed on B lymphocytes and macrophages, but not on other tissue cells. However, many other cell types can be induced to express MHC class II molecules by exposure to cytokines.

Under normal conditions, endogenous peptides (with self determinants potentially leading to autoimmune disease) are not bound to MHC class II molecules since the Ii protein is always cosynthesized with nascent MHC class II molecules. Because complexes containing autodeterminant peptides and MHC class II molecules are never seen by the body's immune surveillance system, tolerance is not developed to these determinants. If MHC class II molecules are not inhibited by Ii in a developed individual, endogenous autodeterminants then become presented by MHC class II molecules, initiating an autoimmune response to those endogenous antigens. Such is the case in certain autoimmune diseases. By engineering such an effect in malignant cells, an “autoimmune response” to the endogenous antigens of a tumor can be used therapeutically to either restrict growth or eliminate tumor cells.

The therapeutic effects of increased MHC class II molecule expression without concomitant increase in Ii protein has been demonstrated in MHC class II-negative, Ii-negative tumors (Ostrand-Rosenberg et al., Journal of Immunol. 144: 4068-4071 (1990); Clements et al., Journal of Immunol. 149: 2391-2396 (1992); Baskar et al., Cell. Immunol. 155: 123-133 (1994); Baskar et al., J. Exp. Med. 181: 619-629 (1995); and Armstrong et al., Proc. Natl. Acad. Sci. USA 94: 6886-6891 (1997)). In these studies, transfection of genes for MHC class II molecules into a MHC class II-negative murine sarcoma generated MHC class II-positive, but Ii-negative tumor cell lines. Injection of these cells into a MHC compatible host led to the delayed growth of the parental tumors. Co-transfection of the gene for the Ii protein into a sarcoma cell line along with the MHC class II genes, inhibited the tumor-therapeutic effect of the MHC class II genes since the Ii chain blocked the presentation of endogenous tumor antigens. Comparable results have been produced with a murine melanoma (Chen and Ananthaswamy, Journal of Immunology 151: 244-255 (1993)).

The success of this therapeutic approach is thought to involve the natural activities of dendritic cells. Dendritic cells are professional scavengers, which process foreign antigens into peptides and present them to T lymphocytes from MHC antigens on their cell surfaces. Dendritic cells have the capacity to present antigen through both MHC class I and class II molecules, enabling them to activate both T helper and T killer cells. It is thought that an effective T helper cell response is required to elicit a powerful T killer cell response and that the combined activation produced by dendritic cells leads to a heightened anti-tumor response (Ridge et al., Nature 193: 474-477 (1998); Schoenberger et al., Nature 193: 480-483 (1998)). The dendritic cells of macrophage lineage, upon finding tumor cells, ingest and process both tumor-specific and tumor-related antigens. The dendritic cells then migrate to the lymph nodes which drain the tumor site and reside in those nodes near the node cortex where new T cells germinate. In the node cortex, resting T killer cells which recognize tumor determinants on the dendritic cells, become activated and proliferate, and are subsequently released into the circulation as competent, anti-tumor, killer T cells.

Although interaction with T-helper cells activates or “licenses” dendritic cells to present antigen through MHC class I molecules, and hence to activate T killer cells, simultaneous interaction with T helper cells and T killer cells is not necessary; activated dendritic cells maintain their capacity to stimulate T killer cells for some time after T helper cell mediated activation. The respective antigenic peptides which become presented by either MHC class II or MHC class I determinants do not need to come from one antigenic protein, two or more antigens from a malignant cell can be processed and presented by a dendritic cell. Therefore, licensing to one determinant, perhaps not tumor specific, carries with it the power to license activation of T killer cells to other, perhaps tumor-specific, determinants. Such ‘minor’ or ‘cryptic’ determinants have been used for various therapeutic purposes (Mougdil et al., J. Immunol. 159: 2574-2579 (1997)).

Experimental alteration of MHC class II antigen presentation is thought to expand immune responses to these minor determinants. The series of peptides usually unavailable for charging to MHC class II molecules, provides a rich source of varied peptides for MHC class II presentation. Exploitation of this series of determinants leads to the expansion of populations of responsive T helper cells. Such expanded populations can elicit dendritic cell licensing, some of which are directed toward tumor specific and tumor related determinants. Although normal cells potentially share tumor cell determinants, only minor cellular damage occurs to normal cells. This is because the multiple effector responses (mass of killer T cells, ambient activating cytokines, phagocytosing macrophages and their products, etc.) of the anti-tumor response is not directed towards normal cells.

Normal MHC class II antigen presentation can be altered by inhibiting the interactions of MHC class II molecules with the Ii protein. This is accomplished by decreasing total Ii protein, (e.g. by decreasing expression) or by otherwise interfering with the Ii immunoregulatory function. Inhibition of Ii expression has been accomplished using various antisense technologies. An antisense oligonucleotide interacting with the AUG site of the mRNA for Ii protein has been described to decrease MHC class II presentation of exogenous antigen (Bertolino et al., Internat. Immunology 3: 435-443 (1991)). However, the effect on the expression of Ii protein and on the presentation of endogenous antigen by MHC class II molecules were not examined. More recently, Humphreys et al., U.S. Pat. No. 5,726,020 (1998) have identified three antisense oligonucleotides and a reverse gene construct which upon introduction into an antigen presenting cell expressing MHC class II molecules expressing effectively suppresses Ii protein expression. Mice inoculated with tumor cells which are Ii suppressed by this mechanism were shown to survive significantly longer than mice inoculated with the untreated parent tumor cells. This observation indicates that the suppression of Ii protein generated an increase in range of antigenic determinant presentation, triggering a more effective immune response to the tumor cells.

In the sarcoma cell (Sal1) tumor model, tumor cells treated with this Ii antisense oligonucleotide are potent vaccine against challenge by parental tumor. As clinically useful in vivo therapeutic antisense reagents, expressible Ii antisense reverse gene constructs (Ii-RGC) were created (U.S. patent application Ser. No. 10/127,347). These were constructed by cloning different Ii gene fragments in reverse orientation into expressible plasmids or adenoviruses, to evaluate multiple methods of tumor cell administration (Hillman et al., Gene Ther. 10, 1512-8 (2003); Hillman et al., Human Gene Therapy 14, 763-775 (2003)). The Ii-RGC genes were evaluated by stable or transient DNA transfections in several murine tumor cell lines, including A20 lymphoma cells, MC-38 colon adenocarcinoma cells, Renca renal adenocarcinoma cells, B16 melanoma cells, and RM-9 prostate cancer cells. The most active one Ii-RGC (−92, 97) (A in the AUG start codon is position 1) was chosen for in vivo studies.

Among the cell lines tested, A20 is already MHC class II+/Ii+. Ii-RGC (−92, 97) significantly inhibited Ii expression when this construct was delivered by lipid or gene gun transfection methods. The other tumor lines tested are MHC class II−/Ii−. These cell lines were co-transfected in vitro with Ii-RGC (−92, 97) and either CIITA or IFN-γ, or both, creating the MHC class II-positive/Ii-suppressed phenotype (Lu et al., Cancer Immunol Immunother 48, 492-8 (2003); Hillman et al., Gene Ther. 10, 1512-8 (2003); Hillman et al. Human Gene Therapy 14, 763-775 (2003)). In vivo induction of the MHC class II-positive/Ii-suppressed phenotype was also generated by intratumoral injection of Ii-RGC and CIITA plasmids with lipid (Lu et al., Cancer Immunol Immunother 48, 492-8 (2003); Hillman et al., Human Gene Therapy 14, 763-775 (2003)) or recombinant adenoviral vectors containing Ii-RGC(−92, 97), CIITA and IFN-γ (Hillman et al., Gene Ther. 10, 1512-8 (2003)).

The in vivo activities of these therapeutic constructs were tested by intratumoral injection in established subcutaneous tumors using two tumor models: the Renca renal carcinoma and the RM-9 prostate carcinoma. In both tumor models, complete regression of established tumors was achieved. In the Renca model, tumor regression was observed in about 50% of mice following four intratumoral injections of CIITA and Ii-RGC plasmid constructs over 4 days given together with a suboptimal dose of IL-2 plasmid (Lu et al., Cancer Immunol Immunother 48, 492-8 (2003)). Intratumoral injections of recombinant adenovirus, containing CIITA, IFN-γ, Ii-RGC constructs and IL-2 gene, in established Renca tumors induced complete tumor regression in about 60-70% of mice and protection against Renca tumor rechallenge (Hillman et al., Gene Ther. 10, 1512-8 (2003)). In an aggressive, poorly immunogenic RM-9 prostate tumor model, radiation augmented the effect of the suboptimal dose of IL-2 and MHC class II-positive/Ii-suppressed phenotype causing complete tumor regression in 50% of the mice (Hillman et al., Human Gene Therapy 14, 763-775, 2003). Established RM-9 subcutaneous tumors were selectively irradiated and treated a day later with intratumoral plasmid gene therapy using the plasmids pCIITA, pIFN-γ, pIL-2 and pIi-RGC for four consecutive days. Intratumoral treatment with all the four plasmids induced complete tumor regression in more than 50% of the mice only when tumor irradiation was administered one day prior to gene therapy. Mice rendered tumor-free by radiation and intratumoral gene therapy and re-challenged on day 64, were protected against RM-9 challenge but not against syngeneic EL4 challenge. These findings demonstrate that in the RM-9 model, radiation enhanced the therapeutic efficacy of intratumoral gene therapy for in situ induction of tumor-specific immune response.

In order to obtain optimal therapeutic effect, MHC class II and Ii must be induced with CIITA and Ii needs to be inhibited by Ii-RGC in both the Renca and RM-9 tumor models (Lu et al., Cancer Immunol Immunother 48, 492-8 (2003); Hillman et al., Human Gene Therapy 14, 763-775, 2003). The results are consistent with those of Martin et al. (J Immunol 162, 6663-70 (1999)) who showed, in a murine lung carcinoma model, that induction of MHC class II by CIITA did not create an efficient tumor cell vaccine. This study confirms our finding that induction of MHC class II by transfecting CIITA, which also induces Ii, is insufficient for a therapeutic effect. One must obtain the therapeutic phenotype of MHC class II+/Ii− by also suppressing Ii protein. In order to test for optimal suppression of Ii protein, the therapeutic constructs CIITA and Ii-RGC were used at different ratios. At least a 1:4 ratio (CIITA:Ii-RGC) was required to ensure good inhibition of Ii. IFN-γ is used in the RM-9 prostate tumor to induce MHC class I molecules which are not expressed in the parental cells. Renca cells are MHC class I-positive cells and IFN-γ is not needed to induce MHC class I molecules but does upregulate further their expression. In both tumor models, a subtherapeutic dose of IL-2 plasmid is needed to promote the immune response.

Given this clear demonstration of efficacy in curing established tumors in mice, and steady progression in preclinical studies to determine optimal treatment protocols, reagents for treating human cancers were created. The CIITA gene we used in the mice studies is human and its product functions well on the murine promoters for MHC class II and Ii genes (Ting et al., Cell 109, 521-33 (1999)). Several human Ii-RGCs, which inhibited Ii expression in a human B lymphoblastoid and the HeLa cell lines were created. Transduction of cells with CIITA construct induced upregulation of cell surface MHC class II molecules and intracellular Ii while transduction of cells with both CIITA and hIi-RGC caused suppression of Ii without affecting enhanced expression of MHC class II. These data were reproduced in additional human tumor cell lines including the human B lymphoma cell line Raji, and human melanoma cell line.

In the present invention, these methods are applied with newly designed RNAi genetic constructs and synthetic oligonucleotides. Double stranded RNA can be used for selective inhibition of target gene expression by RNA interference (RNAi) in mammalian cells. Unlike antisense, RNAi is mediated by the incorporation of double stranded RNA into a nuclease complex, termed the RNA-induced silencing complex (RISC) that subsequently cleaves the target RNA. It has been shown that double stranded RNAs less than 25 nucleotides in length do not activate an RNA response characteristic of viral infections. The RNA sequences can be based on any region of the target gene RNA, generally in the coding region. When using synthetic RNAi, cells are treated in culture using cationic lipids for delivery of nanomolar concentrations of RNAi. Active RNAi may also be engineered into expression constructs. In all studies, RNAi not complimentary to the target sequence is used as a control. Inhibition of gene expression is measured 12 to 72 hours after RNAi treatment using Western, FACS and/or phenotypic assays.

The robust nature of RNAi inhibition of Ii is ideally suited for immune stimulation resulting from the presentation of endogenously synthesized antigens. Ii only needs to be suppressed in a fraction of the cells for a short period of time to obtain immune stimulation. This is in stark contrast to other specific targets related to the growth of cancer cells requiring continuous inhibition in virtually all cells.

RNA interference (RNAi) is a process by which double-stranded RNA (dsRNA) specifically suppresses the expression of a gene bearing its complementary sequence (Moss, Curr. Biol. 11: R772-5 (2001); Elbashir, Genes Dev. 15: 188-200 (2001)). Several gene products have been implicated in this process, including DICER, which is an Rnase that processively cleaves long dsRNA into double-stranded fragments between 21 and 25 nucleotides long. These fragments are known in the art as short interfering or small interfering RNAs (siRNA) (Elbashir et al., 2001).

Studies in Drosophila have shown that DICER processes long dsRNA into siRNAs comprised of two 21 nt strands which includes a 19 nt region on each precisely complementary with the other, yielding a 19 nt duplex region flanked by 2 nt-3′ overhangs (WO 01/75164; Bernstein et al., Nature 409:363, 2001). SiRNAs then induce formation of a protein complex that recognizes and cleaves target mRNAs. Homologs of the DICER enzyme have been identified in species ranging from E. coli to humans (Sharp, 2001; Zamore, Nat. Struct. Biol. 8:746, 2001), suggesting that siRNAs have the ability to silence gene expression in many different cell types including mammalian and human cells.

Subsequently it was discovered that RNAi can be triggered in mammalian cells by introducing synthetic 21-nucleotide siRNA duplexes (Elbashir et al., 2001). In mammalian cell culture, RNAi has been successfully recreated in a wide variety of different cell types with synthetic siRNAs introduced into cells by techniques such as transfection (Elbashir et al., 2001). Because 21 nucleotide siRNAs are too short to induce an interferon response in mammalian cells (Kumar and Carmichael, 1998) but yet long enough to provide sequence specific inhibition of a targeted gene they possess tremendous potential as research tools and therapeutics.

### SUMMARY OF THE INVENTION

The present invention is directed toward composition and methods involving the inhibition of Ii expression in cells for the purpose of altering antigen presentation pathways. The present invention relates in one aspect to siRNAs effective to inhibit Ii expression. In one embodiment, an siRNA of the present invention comprises an RNA duplex. One strand of the RNA duplex contains a sense sequence of Ii. The second strand of the RNA duplex contains a reverse complement of the sense sequence of Ii. In another aspect, the siRNA comprises in a single molecule a sense sequence of Ii, a reverse complement of said sense sequence, and an intervening sequence enabling duplex formation between the sense and reverse complement sequences. In all embodiments, the sense sequence of Ii is preferably 10 to 25 nucleotides in length, more preferably 19 to 25 nucleotides in length, or most preferably 21 to 23 nucleotides in length. In another aspect, the present invention provides DNA sequences which encode siRNAs effective to inhibit Ii expression, cells containing such DNAs or siRNAs, and methods for use of the same.

In one aspect, the invention relates to a method for inhibiting expression of Ii in a cell. This method comprises introducing an siRNA into a cell expressing Ii, wherein the siRNA is introduced either directly or indirectly into the cell. The siRNA thereafter forms an RNA-induced silencing complex, thereby inhibiting expression of Ii in the cell.

The suppression of Ii expression is intended to alter antigen presentation pathways. More specifically, the inhibition of Ii expression is intended to promote the charging of MHC Class II molecules with antigenic epitopes which normally would not be presented in this context. Thus, in another aspect, the present invention relates to the conversion of an MHC Class II molecule-negative cell to an MHC Class II molecule-positive cell. This conversion can be effected, for example, by the transfection of an MHC Class II molecule-negative cell with a recombinant vector comprising an expressible nucleic acid sequence encoding a protein, the transfection of which, in an MHC Class II molecule-negative cell, results in the induction of MHC Class II molecules on the surface of the transfected cell.

In another aspect, the present invention relates to a method for displaying an antigenic epitope of interest on the surface of an MHC Class II molecule-positive cell in which Ii protein expression is suppressed. This method involves: a) providing a cell which is either an MHC Class II molecule-positive cell or is induced to express MHC Class II molecules and which expresses an antigenic epitope of interest; and b) introducing into the cell of step a) an siRNA wherein the siRNA is introduced either directly or indirectly into the cell, and further wherein the siRNA is capable of forming an RNA-induced silencing complex, thereby inhibiting expression of Ii in the cell. In another embodiment, this method may comprise a) providing a cell which is either MHC Class II molecule-positive or is induced to express MHC Class II molecules on its cell surface and further wherein the cell expresses Ii; and b) introducing into the cell of step a) an antigenic epitope of interest and an inhibitor of Ii. The inhibitor of Ii may be an siRNA.

In another aspect, the present invention relates to a method for stimulating an immune response in a mammal, the immune response being directed toward an antigenic epitope of interest on the surface of an MHC Class II molecule-positive cell in which Ii protein expression is suppressed. This method comprises providing either an MHC Class II molecule-positive cell which expresses an antigenic epitope of interest, or an MHC Class II molecule-negative cell which expresses an antigenic epitope of interest and which is induced to express MHC Class II molecules on its cell surface; thereafter introducing into said cell an siRNA wherein the siRNA is introduced either directly or indirectly into the cell, and further wherein the siRNA is capable of forming an RNA-induced silencing complex, thereby inhibiting expression of Ii; and immunizing the mammal with either said cell or an MHC Class II molecule complexed with an antigenic epitope of interest derived from said cell.

In another aspect, the present invention relates to a method for targeting a type of cell of an animal for an immunological response, the type of cell being characterized by the expression of an identifying antigen. In this method a culture of peripheral blood mononuclear cells from an individual is provided, the culture including antigen presenting cells. An siRNA inhibitor of Ii expression is introduced either directly or indirectly into the antigen presenting cell of the culture, as is an expressible nucleic acid sequence encoding the identifying antigen into the cells in the culture under conditions appropriate for expression.

A number of related aspects are described in detail in the following section.

### BRIEF DESCRIPTION OF THE DRAWINGS

FIG. 1 is a diagram representing relative fluorescence intensity. MHC class II+/Ii− phenotype was generated by infection of murine colon adenocarcinoma cells (MC38) with the adeno/CIITA adenoviral vector and subsequent treatment with Ii antisense oligonucleotudes. A) parental MC38 cells (no treatment); B) MC38 cells infected with adeno/CIITA; C) MC38 cells infected with adeno/CIITA and treated with sense control oligonucleotide; D) MC38 cells infected with adeno/CIITA and treated with mismatch control oligonucleotide; E) MC38 cells infected with adeno/CIITA and treated with Ii antisense oligonucleotide.

FIG. 2 is a diagram representing inhibition of MC-38 colon adenocarcinoma growth in mice vaccinated with MHC class II+/Ii− cells. Legend: (circle) mice immunized with MC-38 cells; (triangle) mice immunized with MC-38 cells treated with adeno/CIITA and mismatch control oligonucleotides; (diamond) mice immunized with MC-38 cells treated with adeno/CIITA and sense control oligonucleotides; and (square) mice immunized with MC-38 cells treated with adeno/CIITA and Ii antisense oligonucleotides.

FIG. 3 is a diagram representing inhibition of parental tumor growth in mice inoculated with lethally irradiated MC-38 cells stably transfected with CIITA and inhibited for Ii expression using Ii antisense. Mice were inoculated with CIITA transfected MC-38 cells treated with PBS (triangle), sense oligonucleotide (circle) or Ii antisense (square) (5 mice/group).

FIG. 4 is a diagram representing inhibition of MC-38 colon adenocarcinoma growth in mice vaccinated with MHC class II+/Ii− cells and treated with GM-CSF. Legend: (triangle) mice immunized with parental MC-38 cells; (circle) mice immunized with MC-38 cells and GM-CSF; (open square) mice immunized with MC-38 cells treated with CIITA, sense control oligonucleotides and GM-CSF; and (diamond) mice immunized with MC-38 cells treated with CIITA, Ii antisense oligonucleotides and GM-CSF.

FIG. 5 is a diagram representing MHC class II molecule and Ii induction by adeno/IFN-.gamma. in MC-38 cells. MC-38 cells were infected with adeno/IFN-.gamma. (3 MOI) for the times indicated, then stained with anti-MHC class II molecule or Ii antibodies and analyzed by flowcytometry.

FIG. 6 is a diagram representing relative fluorescence intensity. MHC class II+/Ii− phenotype was generated in Renca cells by co-infection of cells with adeno/CIITA and adeno/Ii-RGC (Ii-92, 97). Renca cells were co-infected with different ratios of adeno/CIITA to adeno/Ii-RGC, allowed to incubate for 72 hours and stained for MHC class II molecule or Ii protein expression. Parental Renca cells are shown in A; adeno/CIITA infected cells in B; co-infection with adeno/CIITA to adeno/Ii-RGC at a 1:2 ratio in C; and co-infection with adeno/CIITA to adeno/Ii-RGC at a 1:4 ratio in D.

FIG. 7 is a representation of a time course experiment in which MHC class II+/Ii− phenotype was generated in MC-38 cells by infection of cells with adeno/IFN-.gamma./Ii-RGC(mIi-92, 97). An Ii− but class II+ phenotype has been created at 120 hour after adeno/IFN-.gamma./Ii-RGC(mIi-−92, 97) (left) while infection with the adeno/IFN-.gamma. alone did not produce the MHC class II+/Ii− phenotype in MC-38 cells (right).

FIG. 8 is a representation of Ii-suppression in transiently transfected Raji cells (MHC class II+/Ii+), a human B-lymphoma cell line. Cells were plated into a 12-well plate overnight at 1.25.times.10.sup.5 cells/well and transfected with human Ii-reverse gene constructs (hIi-RGC) to inhibit Ii expression. Effectene transfection reagent (25.mu.l, QIAGEN) was incubated with condensed hIi-RGC plasmid DNA (1 ug) to produce effectene DNA complexes mixed with medium which was directly added to the cells. After 48 hours incubation, the cells were analyzed for Ii and MHC-class II molecule expression by immunostaining with anti-human Ii antibody, LN2 (Pharmingen) and anti-HLA-DR antibody for staining of MHC class II molecules (Pharmingen). As can be seen, Ii expression was inhibited in 4% and 9% of the cells, depending on the Ii-RGC sequence used, compared to positive control cells (left panel), while there was no effect on MHC-class II molecule expression (right panel)

FIG. 9 is a representation of inhibition of tumor growth by in vivo administration of the adeno/Ii-RGC vector and generation of the MHC class II+/Ii− phenotype. BALB/c mice were injected subcutaneously with 5.times.10.sup.5 Renca renal adenocarcinoma cells. When the tumors reached a size between 50-200 mm.sup.3, about 10 days after tumor cell injection, the tumors were injected with different vector combinations on each of four consecutive days with DMRIE/c. The tumors were then measured every two or three days for the size. Mice were terminated when tumor sizes reaches 1000 mm.sup.3. The data on the left panel represent four mice whose tumors were injected with 2.mu.g IL-2, 3.mu.g adeno/BN/CIITA and 18.mu.g adeno/BN/Ii-RGC(−92, 97) on day 1 followed by 2.mu.g IL-2, 18.mu.g adeno/BN/Ii-RGC(−92, 97) and 3 μg empty plasmid (adeno/BN) (without CIITA) for days 2-4. It is clear that mice treated with CIITA and Ii-RGC containing vectors together with IL-2 exhibited a dramatic reduction in tumor growth, while tumor growth in mice receiving only IL-2 and control vector was progressive and required termination of the mice.

### DETAILED DESCRIPTION OF THE INVENTION

The subject invention relates, in one aspect, to compositions and methods for pathology-specific modulation, or targeting, of the immune response in an individual. Modulation, as that term is used herein, is meant to refer to increased sensitivity or decreased sensitivity (tolerance) of the immune system in an individual to an antigen. Targeting, as that term is used, is intended to refer to increased sensitivity to an antigenic epitope.

A required element relating to all aspects of the present disclosure is the inhibition of Ii synthesis in a cell. The term “inhibition” or “suppression” is intended to mean down regulation, or the act of reducing the activity of Ii or level of Ii RNAs below that observed in the absence of an inhibitor or suppressor of the present invention. As discussed in the Background of the Invention section, Ii is a protein, which is co-regulated with the MHC Class II molecules. Ii binds MHC Class II molecules thereby blocking access to MHC Class II molecules of endogenously synthesized antigens (i.e., antigen synthesized within the MHC Class II molecule-expressing cells). The MHC Class II molecule/Ii complexes are transported from the endoplasmic reticulum to a post-Golgi compartment where Ii is released by a staged cleavage process which enables charging by exogenous antigen (i.e., antigen which is not synthesized within the antigen presenting cell and has been selected for uptake into the antigen presenting cell by mechanisms such as phagocytosis, opsonization, cell surface antibody recognition, complement receptor recognition, and Fc receptor recognition).

The class of antigen excluded from binding to MHC Class II molecules in the endoplasmic reticulum by virtue of the presence of complexed Ii protein can be referred to as endogenously synthesized antigen. Such antigen comprises a survey of cytoplasmic proteins, which have been digested by proteosomes and transported as peptides into the endoplasmic reticulum by the transporter of antigenic peptides (TAP). Such endogenously synthesized antigen is normally bound to MHC Class I molecules in the endoplasmic reticulum. Such antigenic fragments are not normally bound in the endoplasmic reticulum to MHC Class II molecules because Ii protein blocks the antigenic peptide-binding site.

By suppressing the expression of the Ii protein, this vast repertoire of peptides which have been transported into the endoplasmic reticulum for binding to MHC Class I and subsequent presentation to CD8+ T lymphocytes, can bind to MHC Class II molecules for subsequent presentation to, and activation of, CD4+ T immunoregulatory cells. Such CD4+ T immunoregulatory cells can have either helper or suppressor functions in orchestrating various pathways of the immune response. T immunoregulatory cells contribute to the activation of other cells, such as cytotoxic T lymphocytes (T killer cells), B lymphocytes, and dendritic cells, via physical contact and cytokine release.

The term “antigenic epitope of interest”, as used herein, refers to an antigenic epitope present in a peptide derived from a protein produced within the cell on which antigen presentation is to take place. The term, as used herein, is intended to encompass antigenic epitopes which are known or unknown. Thus the modifier “of interest” does not imply that the epitope is predetermined. An antigenic epitope is “of interest” merely by virtue of the fact that is contained in a protein which is synthesized in the cytoplasm of the cell on which presentation is to take place.

A significant biological consequence offering an opportunity for therapeutic intervention, follows from the binding by MHC Class II molecules of peptides from the repertoire of peptides transported into the endoplasmic reticulum for binding there by MHC Class I molecules. Often the epitopes bound to the MHC Class II molecules in the presence of Ii suppression are “cryptic” epitopes in that such epitopes are not otherwise presented to the immune system in association with MHC Class II molecules by classical pathways of antigen presentation. Cryptic epitopes can be revealed experimentally by analyzing a library of overlapping synthetic peptides of the amino acid sequence of a test antigen. Animals of one strain of mice immunized with the test antigen can be found to respond to a set of peptides from the library (the “dominant epitopes”). However, when otherwise identical mice are immunized with single peptides of the library, a previously unidentified subset (in addition to any dominant epitopes in the immunizing peptide) is found to contain immunological epitopes. These previously unidentified epitopes comprise a set of cryptic epitopes.

Although the method of this invention promotes immunity against both dominant and cryptic epitopes, in some clinical situations the enhancement of the immune response to cryptic epitopes plays a special role in the therapeutic effect. For example, in the case of boosting a therapeutic response to cancer-related antigenic epitopes, a T helper cell response to cryptic epitopes to which a suppressor T cell response had never occurred is more likely to provide for effective dendritic cell licensing which, in turn, creates a robust cytotoxic T lymphocyte anti-tumor response. The development of suppressing T cell responses to dominant epitopes of cancer-related antigens has been indicated to play a role in the growth of tumor micrometastases. A significant utility of this invention is therefore promotion of T helper cell responses to putatively cryptic cancer-related determinants.

In another aspect, in the case of autoimmune disease, a response to dominant epitopes of autoimmune disease-related antigens promotes the pathogenesis of such diseases. Here the exploitation of alternative, e.g. suppressing, pathways of immune response to novel cryptic epitopes can be therapeutically useful. These concepts can likewise be applied in the therapy of additional medical conditions such as allergy, graft rejection, and infectious and cardiovascular diseases. An essential and useful first step in the development of compounds to be applied in the diagnosis, treatment monitoring, and therapy of patients with such conditions, is the identification of MHC Class II epitopes which become presented by antigen presenting cells under the condition of Ii protein suppression. Such epitopes include both dominant and cryptic epitopes. Cryptic epitopes may be particularly useful since imunosuppressing responses will not have been developed toward them, for example, in the case of cancer or infectious disease, and activating responses will not have been developed in the case of autoimmune diseases or graft rejection. The clinician therefore has a fresh start in eliciting a Th1 or Th2, activating or suppressing, response as the case might be in a given pathological condition. Methods to generate, isolate and characterize such epitopes are a subject of this invention.

In another aspect, the invention provides for presentation, isolation and identification of individual peptides containing antigenic epitopes, which are bound to MHC Class II molecules in the endoplasmic reticulum in the absence of the Ii protein. Such peptides can be synthesized and used individually or in combination in vaccine applications to enhance or suppress immune responses to the disease-related antigens from which they originated. The methods to isolate and characterize such epitope peptides have been presented in U.S. Pat. No. 5,827,526, U.S. Pat. No. 5,874,531, and U.S. Pat. No. 5,880,103, which are incorporated herein by reference.

A variety of antigens which fall within the “endogenously synthesized” class (which are normally excluded from MHC Class II molecule presentation) are specifically associated with certain pathological conditions. Consider, for example, tumor cells or other malignant cells. Such cells synthesize cancer-specific and cancer-related proteins, which contain therapeutically useful MHC Class II epitopes. However, because these proteins are synthesized within the antigen presenting cell, antigenic epitopes of such proteins are excluded from presentation in association with MHC Class II molecules of the same cell. This restriction on the presentation of antigenic epitopes by the cell, in which the antigenic protein is synthesized, holds also in the case of virally infected cells. Virus-specific antigens are excluded from presentation in association with MHC Class II molecules of the virus-infected cell, while those antigens can be presented in association with MHC Class I molecules of the same cell. To the extent that other exogenous pathogens (e.g., a bacterium or parasite) occupies a cell and utilizes cellular machinery to synthesize protein specific to the pathological condition, the issues are the same. The ability to alter the normal course of events, thereby presenting pathology-specific antigen in association with MHC Class II molecules, results in enhancement of responses initiated by novel MHC Class II antigenic epitopes.

An array of therapeutic modalities fall within the scope of the present invention. Patentable compositions are associated with many of these therapeutic modalities. Therapeutic approaches include in vivo and ex vivo embodiments. Cells which are targeted for Ii inhibition can be either MHC Class II molecule-positive cells (e.g., naturally occurring, antigen presenting cells such as dendritic cells, macrophages or B lymphocytes), or MHC Class II molecule-negative cells (e.g., tumor cells) which are induced to express MHC Class II molecules. The expression “MHC Class II molecule-negative”, as used herein, specifically includes not only cells which express no MHC Class II molecules on their cell surface, but also cells containing a relatively low number of MHC Class II molecules on their cell surface when compared to the number of MHC Class II molecules on the surface of a positive control cell such as a naturally occurring antigen presenting cell (e.g., a dendritic cell). The term “relatively low”, in this context, is meant to include cells estimated to contain only about 25%, or less, of the number of MHC Class II molecules on their cells surface as would be found on an MHC Class II molecule-positive control cell (e.g., a naturally occurring antigen presenting cell). The estimate of MHC Class II molecule abundance can be made, for example, using immunofluorescent techniques which are well known in the art.

Applicants have previously filed and prosecuted patent applications disclosing Ii inhibition for the purpose of modulating the immune response. These applications specifically disclose inhibitory copolymers which are introduced into a cell and which directly inhibit Ii synthesis by binding to the Ii mRNA, as well as reverse gene constructs which are introduced into a cell as a nucleic acid construct which is subsequently transcribed into an RNA molecule which inhibits Ii expression after specific hybridization. These earlier filed patent applications include U.S. application Ser. Nos. 08/661,627, 09/205,995, 10/054,387 and 10/127,347, the disclosures of which are incorporated herein by reference. U.S. application Ser. Nos. 08/661,627 and 09/205,995 have issued as U.S. Pat. Nos. 5,726,020 and 6,368,855, respectively.

As mentioned briefly above, U.S. application Ser. No. 09/205,995 contains extensive disclosure relating to chemically synthesized copolymers containing from about 10 to about 50 nucleotide bases. These copolymers contain nucleotide base sequences which are complementary to a targeted portion of the RNA molecule, otherwise known as antisense sequences. Examples of such copolymers include antisense oligonucleotides and siRNAs. Antisense copolymers inhibit protein translation from RNA by two mechanisms. One method is to block access to portions of the RNA which must interact with ribosomes, spliceosomes or other factors essential for RNA maturation or translation. A second method, involves potentiation of an enzyme, ribonuclease H, which cleaves sequences of RNA hybridized to DNA. Thus, the binding of a DNA or DNA like copolymer to a corresponding segment in the RNA leads to cleavage of the RNA at the copolymer binding site.

Copolymers hybridize to the target RNA, such as by Watson-Crick base pairing. The sequence of a copolymer is defined by the complementary sequence of the target RNA. The copolymers are usually synthesized chemically with nucleotide sequence lengths which span at least 6 complementary nucleotides of the target RNA, with 12-25 being most common. Statistically, a sequence of about 15 nucleotides is unique within the population of all RNAs within a cell, enabling any particular RNA to be targeted with a high degree of specificity. Binding to RNA is also very stable with Kd values around 10⁻¹⁷ M, for a copolymer encompassing 20 base pairs.

In some cases, cells in culture spontaneously take up copolymers in a sufficient amount to achieve a useful effect. Such uptake appears to be an active process requiring biochemical energy and participation of certain cell surface proteins. Uptake can also occur by pinocytosis. This route can be enhanced by incubating cells in a hypertonic medium containing a copolymer followed by resuspension of the cells in a slightly hypotonic medium to induce bursting of intracellular pinocytotic vesicles. In other cases, uptake can be assisted by use of lipids, liposomes, or polyalkyloxy copolymers, by electroporation, or by streptolysin O treatment to permeabilize the cell membrane. Cells in vivo often take up copolymers more readily than do cultured cells. Optimal conditions for cell uptake of copolymers by electroporation are provided in Example 2 of U.S. application Ser. No. 09/205,995.

Potential sites of the target RNA are those open for binding of functional complexes of proteins, and additional sites which are otherwise open for copolymer binding. Such sites can be identified using ribonuclease H (RNase H), an enzyme which cleaves RNA that is hybridized to DNA. By adding DNA oligonucleotides, singly or in mixtures, to 5′-radiophosphorus-labeled RNA in the presence of ribonuclease H, the sites on the RNA where oligonucleotides and other copolymers hybridize are identified after gel electrophoresis of the RNA and autoradiography. The sites in the Ii RNA found in the present invention to be most open for RNase H cleavage, were the region of the AUG initiator codon and the region of the first splice site in the pre-mRNA.

The term “oligonucleotide” regarding the present invention refers to polynucleotides comprising nucleotide units formed with naturally occurring bases and pentofuranosyl sugars joined by phosphodiester linkages. The term “copolymer” includes oligonucleotides and also structurally related molecules formed from non-naturally occurring or modified subunits of oligonucleotides. These modifications occur either on the base portion of a nucleotide, on the sugar portion of a nucleotide, or on the internucleotide linkage groups. Additional linkage groups are often also substituted for sugar and phosphate backbone of a natural oligonucleotide to generate a copolymer, discussed in greater detail below.

Such oligonucleotide modifications and the characteristics which are produced are readily available to one of skill in the art. Exemplary modifications are presented in U.S. Pat. No. 4,469,863 (1984); U.S. Pat. No. 5,216,141 (1993); U.S. Pat. No. 5,264,564 (1993); U.S. Pat. No. 5,514,786 (1996); U.S. Pat. No. 5,587,300 (1996); U.S. Pat. No. 5,587,469 (1996); U.S. Pat. No. 5,602,240 (1997); U.S. Pat. No. 5,610,289 (1997); U.S. Pat. No. 5,614,617 (1997); U.S. Pat. No. 5,623,065 (1997); U.S. Pat. No. 5,623,070 (1997); U.S. Pat. No. 5,700,922 (1997); and U.S. Pat. No. 5,726,297 (1998), the disclosures of which are incorporated herein by reference.

The ability of oligonucleotides to hybridize to complementary RNA is very tolerant of chemical modifications. Therefore, many different functional copolymers are possible. The sugar phosphate backbone, in particular, can be altered extensively without losing the ability to form Watson-Crick base pairs. By definition, a nucleotide comprises a sugar, nitrogen heterocycle and phosphate moieties. Some synthetic analogues of oligonucleotides lack either a sugar or phosphate group or both yet still can hybridize by Watson-Crick base pairs in the same way as antisense oligonucleotides and can be used for the same purposes. These copolymers containing nucleotide bases are functional equivalents of oligonucleotides in hybridizing to RNA. Summarized below are some of the modifications to oligonucleotides which change and improve their properties for antisense applications.

A large number of specific modifications were disclosed, including replacement of non-bridging oxygen atoms, replacement of bridging oxygen atoms, replacement of internucleoside phosphate groups, changes to stereochemistry around the sugar ring, ribofuranosyl ring structure modification, nucleotide linkage modification, and sugar phosphate backbone replacement by a peptide backbone to yield a peptidyl nucleic acid (pna). Prosecution of the referenced patent application resulted in the allowance of the following claim, which is a representative independent claim of U.S. Pat. No. 6,368,855.

1. An MHC class II-positive antigen presenting cell which does not contain an exogenous construct encoding mammalian B7 molecule, and which contains a specific regulator of Ii protein expression or immunoregulatory function, the oligonucleotide CTCGGTACCTACTGG being specifically excluded, the specific regulator consisting essentially of a copolymer of from 10 to 50 nucleotide bases, the copolymer being characterized by the ability to hybridize specifically to a target region of the RNA molecule encoding mammalian Ii protein under physiological conditions, wherein the specific regulator is characterized by the ability to inhibit Ii expression.

For purposes of providing relevant background information, and support for additional claim limitations, it is noted that at least two specific limitations in the claim set forth above were included to address prior art disclosures. For example, the specific exclusion of the oligonucleotide 3′ CTCGGTACCTACTGG 5′ was incorporated in light of the disclosure of Bertolino et al., Int. Immunol. 3(5): 435-443 (1991). The limitation directed toward an exogenous construct encoding mammalian B7 molecule was introduced in light of the disclosure of Ostrand-Rosenberg (U.S. Pat. No. 5,858,776).

An important element of the present disclosure, which has not been previously published, is the use of a reverse gene construct to inhibit the expression of Ii in human cells. While the human Ii sequence has been previously reported (Strubin et al., EMBO J. 3: 869-872 (1984)), the use of a reverse gene construct containing at least a portion of this sequence had never been reported. Furthermore, although significant conservation between, for example, the murine Ii sequence and the human Ii sequence has been reported, non-human reverse gene constructs have been ineffective for use in the inhibition of translation of Ii in human cells.

Thus, in one aspect, the present invention relates to an expressible reverse gene construct, comprising a DNA molecule which encodes an RNA molecule which is complementary to an mRNA molecule which encodes human Ii protein, the RNA molecule having the ability to hybridize with the mRNA molecule thereby inhibiting translation of the mRNA molecule in a human cell. This aspect of the invention is specifically demonstrated in the Exemplification section which follows. More specifically, it was demonstrated that expression constructs containing cDNA inserts were effective in inhibiting Ii expression in a human lymphoma cell line. Constructs which were effective in this assay included cDNA inserts complementary to a portion of the Ii mRNA 5′ untranslated region and included the translation initiation codon. Effective constructs encoded an inhibitory RNA of up to about 435 nucleotides in length.

In addition to the use of reverse gene constructs that encode RNAs which are perfectly complementary with portions of the human Ii mRNA, one of skill in the art will recognize that some degree of divergence from wild-type human sequence will be tolerated. The scope of the present invention is intended to encompass such variants that can be determined empirically by routine experimentation (i.e., they will be characterized by the ability to inhibit Ii expression in a human cell). An example of a variation from wild-type which is particularly useful, and which was demonstrated to be effective in inhibiting Ii expression in human cells, relates to the creation of a long half-life antisense RNA (relative to wild-type antisense RNA) complementary to human Ii mRNA. In the long-half life species, the reading frame of the antisense RNA is designed to avoid the occurrence of the initiation codon, AUG, followed shortly/immediately in the same reading frame by a stop codon. To avoid this situation, for example, with respect to an AUG occurring shortly before a stop codon in reading frame 1, a new AUG can be designed and introduced prior to the AUG of reading frame 1, in either reading frame 2 or reading frame 3, provided that no stop codon occurs in that reading frame after that modification.

In addition to the use of Ii reverse gene constructs, it will be recognized by those skilled in the art that other inhibitory copolymers of Ii expression are readily designed and constructed. For example, double-stranded small interfering RNAs (siRNAs), and genes encoding these molecules, may be used to inhibit Ii by RNA interference.

It is an object of the present invention to provide a composition comprising an siRNA effective to inhibit Ii expression, vectors and cells containing such compositions, and methods of use for the same.

The term “RNA interference (RNAi)” as used herein refers to the process by which double-stranded RNA (dsRNA) specifically suppresses the expression of a gene bearing its complementary sequence (Moss, Curr. Biol. 11(19): R772-5 (2001); Elbashir, Genes Dev. 15(2): 188-200 (2001)). While not wishing to be bound by theory, RNAi is understood to occur by a mechanism involving multiple RNA-protein interactions, characterized by four major steps: assembly of siRNA with the RNA-induced silencing complex (RISC), activation of the RISC, target recognition and target cleavage. The term “short interfering RNAs (siRNA)” as used herein is intended to refer to any nucleic acid molecule capable of mediating RNAi or gene silencing. The term siRNA is intended to encompass various naturally generated or synthetic compounds, with RNAi function. Such compounds include, without limitation, duplex synthetic oligonucleotides, of about 21 to 23 base pairs with terminal overlaps of 2 or 3 base pairs; hairpin structures of one oligonucleotide chain with sense and complementary, hybridizing, segments of 21, -23 base pairs joined by a loop of 3-5 base pairs; and various genetic constructs leading to the expression of the preceding structures or functional equivalents. Such genetic constructs are usually prepared in vitro and introduced in the test system, but can also include siRNA from naturally occurring siRNA precursors coded by the genome of the host cell or animal.

It is not a requirement that an siRNA of the present invention be comprised solely of RNA. An siRNA of the present invention may comprise one or more chemical modifications and/or nucleotide analogues. The modification and/or analogue may be any modification and/or analogue, respectively, that does not negatively affect the ability of the siRNA to inhibit Ii expression. The inclusion of one or more chemical modifications and/or nucleotide analogues in an siRNA may be preferred to prevent or slow nuclease digestion, and in turn, create a more stable siRNA for practical use. Chemical modifications and/or nucleotide analogues which stabilize RNA are known in the art. Phosphorothioate derivatives, which include the replacement of non-bridging phosphoroyl oxygen atoms with sulfur atoms, are one example of analogues showing increased resistance to nuclease digestion. Sites of the siRNA which may be targeted for chemical modification include the loop region of a hairpin structure, the 5′ and 3′ ends of a hairpin structure (e.g. cap structures), the 3′ overhang regions of a double-stranded linear siRNA, the 5′ or 3′ ends of the sense strand and/or antisense strand of a linear siRNA, and one or more nucleotides of the sense and/or antisense strand.

As used herein, the term siRNA is intended to be equivalent to any term in the art defined as a molecule capable of mediating sequence-specific RNAi. Such equivalents include, for example, double-stranded RNA (dsRNA), micro-RNA (mRNA), short hairpin RNA (shRNA), short interfering oligonucleotide, and post-transcriptional gene silencing RNA (ptgsRNA).

While not wishing to be bound by theory, it is generally understood that in RNAi double-stranded RNA is processed into 21 to 23 base-pair fragments that bind to and lead to the degradation of the complementary mRNA (Bernstein, Nature 409(6818): 363-6 (2001), and International Publication Number WO 0175164). siRNAs induce sequence-specific posttranslational gene silencing. Such molecules may be introduced into cells to suppress gene expression for therapeutic or prophylactic purposes as described in International Publication Number WO 0175164. Such molecules may be introduced into cells to suppress gene expression for therapeutic or prophylactic purposes as described in various patents, patent applications and papers. Publications herein incorporated by reference, describing RNAi technology include but are not limited to the following: U.S. Pat. No. 6,686,463, U.S. Pat. No. 6,673,611, U.S. Pat. No. 6,623,962, U.S. Pat. No. 6,506,559, U.S. Pat. No. 6,573,099, and U.S. Pat. No. 6,531,644; International Publication Numbers WO04061081; WO04052093; WO04048596; WO04048594; WO04048581; WO04048566; WO04046320; WO04044537; WO04043406; WO04033620; WO04030660; WO04028471; WO 0175164. Papers which describe the methods and concepts for the optimal use of these compounds include but are not limited to the following: Brummelkamp Science 296: 550-553 (2002); Caplen Expert Opin. Biol. Ther. 3:575-86 (2003); Brummelkamp, Sciencexpress 21 Mar. 3 1-6 (2003); Yu Proc Natl Acad Sci USA 99:6047-52 (2002); Paul Nature Biotechnology 29:505-8 (2002); Paddison Proc Natl Acad Sci USA 99:1443-8 (2002); Brummelkamp Nature 424: 797-801 (2003); Brummelkamp, Science 296: -550-3 (2003); Sui Proc Natl Acad Sci USA 99: 5515-20 (2002); Paddison, Genes and Development 16:948-58 (2002).

In the context of the present invention, a composition comprising an siRNA effective to inhibit Ii expression may include an RNA duplex comprising a sense sequence of Ii. In this embodiment, the RNA duplex comprises a first strand comprising a sense sequence of Ii and a second strand comprising a reverse complement of the sense sequence of Ii. In one embodiment the sense sequence of Ii comprises of from 10 to 25 nucleotides in length. More preferably, the sense sequence of Ii comprises of from 19 to 25 nucleotides in length. Most preferably, the sense sequence of Ii comprises of from 21 to 23 nucleotides in length. The sense sequence of Ii preferably comprises a sequence of Ii containing a translational start site, and more preferably comprises a portion of Ii sequence within the first 400 nt of the human Ii mRNA.

In another embodiment, a composition comprising an siRNA effective to inhibit Ii expression may comprise in a single molecule a sense sequence of Ii, the reverse complement of the sense sequence of Ii, and an intervening sequence enabling duplex formation between the sense and reverse complement sequences. The sense sequence of Ii may comprise 10 to 25 nucleotides in length, or more preferably 19 to 25 nucleotides in length, or most preferably 21 to 23 nucleotides in length. An siRNA of the present invention may comprise the RNA of a sequence selected from the group consisting of SEQ ID NO: 11, SEQ ID NO: 12, SEQ ID NO: 13, SEQ ID NO: 14, SEQ ID NO: 15, SEQ ID NO: 16, SEQ ID NO: 17 and SEQ ID NO: 18.

It will be readily apparent to one of skill in the art that an siRNA of the present invention may comprise a sense sequence of Ii or the reverse complement of the sense sequence of Ii which is less than perfectly complementary to each other or to the targeted region of Ii. In other words, the siRNA may comprise mismatches or bulges within the sense or reverse complement sequence. In one aspect, the sense sequence or its reverse complement may not be entirely contiguous. The sequence or sequences may comprise one or more substitutions, deletions, and/or insertions. The only requirement of the present invention is that the siRNA sense sequence possess enough complementarity to its reverse complement and to the targeted region of Ii to allow for RNAi activity. It is an object of the present invention, therefore, to provide for sequence modifications of an siRNA of the present invention that retain sufficient complementarity to allow for RNAi activity. One of skill in the art may predict that a modified siRNA composition of the present invention will work based on the calculated binding free energy of the modified sequence for the complement sequence and targeted region of Ii. Calculation of binding free energies for nucleic acids and the effect of such values on strand hybridization is known in the art.

A wide variety of delivery systems are available for use in delivering an siRNA of the present invention to a target cell in vitro and in vivo. An siRNA of the present invention may be introduced directly or indirectly into a cell in which Ii inhibition is desired. An siRNA may be directly introduced into a cell by, for example, injection. As such, it is an object of the invention to provide for a composition comprising an siRNA effective to inhibit Ii in injectable, dosage unit form. An siRNA of the present invention may be injected intravenously or subcutaneously as an example, for therapeutical use in conjunction with the methods and compositions of the present invention. Such treatment may include intermittent or continuous administration until therapeutically effective levels are achieved to inhibit Ii expression in the desired tissue.

Indirectly, an expressible DNA sequence or sequences encoding the siRNA may be introduced into a cell, and the siRNA thereafter transcribed from the DNA sequence or sequences. It is an object of the present invention, therefore, to provide for compositions comprising a DNA sequence or sequences which encode an siRNA effective to inhibit Ii expression.

A DNA composition of the present invention comprises a first DNA sequence which encodes a first RNA sequence comprising a sense sequence of Ii and a second DNA sequence which encodes a second RNA sequence comprising the reverse complement of the sense sequence of Ii. The first and second RNA sequences, when hybridized, form an siRNA duplex capable of forming an RNA-induced silencing complex, the RNA-induced silencing complex being capable of inhibiting Ii expression. The first and second DNA sequences may be chemically synthesized or synthesized by PCR using appropriate primers to Ii. Alternatively, the DNA sequences may be obtained by recombinant manipulation using cloning technology, which is well known in the art. Once obtained, the DNA sequences may be purified, combined, and then introduced into a cell in which Ii inhibition is desired. Alternatively, the sequences may be contained in a single vector or separate vectors, and the vector or vectors introduced into the cell in which Ii inhibition is desired.

Delivery systems available for use in delivering a DNA composition of the present invention to a target cell include, for example, viral and non-viral systems. Examples of suitable viral systems include, for example, adenoviral vectors, adeno-associated virus, lentivirus, poxvirus, retroviral vectors, vaccinia, herpes simplex virus, HIV, the minute virus of mice, hepatitis B virus and influenza virus. Non-viral delivery systems may also be used, for example using, uncomplexed DNA, DNA-liposome complexes, DNA-protein complexes and DNA-coated gold particles, bacterial vectors such as salmonella, and other technologies such as those involving VP22 transport protein, Co-X-gene, and replicon vectors. A viral or non-viral vector in the context of the present invention may express the antigen of interest.

One option for expressing a nucleic acid sequence of interest in an animal cell is the adenovirus system. In the Exemplification section which follows, the use of an adenovirus system is specifically disclosed. Adenovirus possesses a double-stranded DNA genome, and replicates independently of host cell division. Adenoviral vectors offer a variety of advantages relative to alternative methods for introducing expressible constructs into cells. For example, adenoviral vectors are capable of transducing a broad spectrum of human tissues and high levels of gene expression can be obtained in dividing and nondividing cells. Adenoviral vectors are characterized by a relatively short duration of transgene expression due to immune system clearance and dilutional loss during target cell division. Several routes of administration can be used including intravenous, intrabiliary, intraperitoneal, intravesicular, intracranial and intrathecal injection, and direct injection of a target organ or tissue. Thus, it is recognized in the art that targeting based on anatomical boundaries is achievable.

The adenoviral genome encodes about 15 proteins and infection involves a fiber protein which binds to a cell surface receptor. This receptor interaction results in internalization of the virus. Viral DNA enters the nucleus of the infected cell and transcription is initiated in the absence of cell division. Expression and replication is under control of the E1A and E1B genes (see Horwitz, M. S., In Virology, 2.sup.nd ed., 1990, pp. 1723-1740). Removal of E1 genes renders the virus replication-incompetent.

Adenoviral serotypes 2 and 5 have been extensively used for vector construction. Beft et al. (Proc. Nat. Acad. Sci. U.S.A. 91: 8802-8806 (1994)) have used an adenoviral type 5 vector system with deletions of the E1 and E3 adenoviral genes. The 293 human embryonic kidney cell line has been engineered to express E1 proteins and can thus transcomplement the E1-deficient viral genome. The virus can be isolated from 293 cell media and purified by limiting dilution plaque assays (Graham and Prevek, In Methods in Molecular Biology: Gene Transfer and Expression Protocols, Humana Press 1991, pp. 109-128). Recombinant virus can be grown in 293 cell line cultures and isolated by lysing infected cells and purification by cesium chloride density centrifugation. A problem associated with the 293 cells for manufacture of recombinant adenovirus is that due to additional flanking regions of the E1 genes, they may give rise to replication competent adenovirus (RCA) during the viral particle production. Although this material is only wild-type adenovirus, and is not replication competent recombinant virus, it can have significant effects on the eventual yield of the desired adenoviral material and lead to increased manufacturing costs, quality control issues for the production runs and acceptance of batches for clinical use. Alternative cell lines such as the PER.C6 which have more defined E1 gene integration than 293 cells (i.e. contain no flanking viral sequence) have been developed which do not allow the recombination events which produce RCA and thus have the potential to overcome above viral production issues.

Adeno-associated virus (AAV) (Kotin, R. M., Hum. Gene Ther. 5: 793-801 (1994)) are single-stranded DNA, nonautonomous parvoviruses able to integrate into the genome of nondividing cells of a very broad host range. AAV has not been shown to be associated with human disease and does not elicit an immune response.

AAV has two distinct life cycle phases. Wild-type virus will infect a host cell, integrate and remain latent. In the presence of adenovirus, the lytic phase of the virus is induced, which depends on the expression of early adenoviral genes, and leads to active virus replication. The AAV genome is composed of two open reading frames (called rep and cap) flanked by inverted terminal repeat (ITR) sequences. The rep region encodes four proteins which mediate AAV replication, viral DNA transcription, and endonuclease functions used in host genome integration. The rep genes are the only AAV sequences required for viral replication. The cap sequence encodes structural proteins that form the viral capsid. The ITRs contain the viral origins of replication, provide encapsidation signals, and participate in viral DNA integration. Recombinant, replication-defective viruses that have been developed for gene therapy lack rep and cap sequences. Replication-defective AAV can be produced by co-transfecting the separated elements necessary for AAV replication into a permissive 293 cell line. U.S. Pat. No. 4,797,368 contains relevant disclosure and such disclosure is incorporated herein by reference.

Retroviral vectors are useful for infecting dividing cells, and are composed of an RNA genome that is packaged in an envelope derived from host cell membrane and viral proteins. Retroviral gene expression involves a reverse transcription step in which its positive-strand RNA genome is employed as a template to direct the synthesis of double-stranded DNA, which is then integrated into the host cell DNA. The integrated provirus is able to use host cell machinery for gene expression.

Murine leukemia virus is a commonly employed retrovirus species (Miller et al., Methods Enzymol. 217: 581-599 (1993)). Retroviral vectors are typically constructed by deletion of the gag, pol and env genes. The deletion of these sequences provides capacity for insertion of nucleic acid sequences of interest, and eliminates the replicative functions of the virus. Genes encoding antibiotic resistance often are included as a means of selection. Promoter and enhancer functions also may be included, for example, to provide for tissue-specific expression following in vivo administration. Promoter and enhancer functions contained in long terminal repeats may also be used.

Such viruses, and modifications of such viruses which carry an exogenous nucleic acid sequence of interest, can only be produced in viral packaging cell lines. The packaging cell line may be constructed by stably inserting the deleted viral genes (gag, pol and env) into the cell such that they reside on different chromosomes to prevent recombination. The packaging cell line is used to construct a producer cell line that will generate replication-defective retrovirus containing the nucleic acid sequence of interest by inserting the recombinant proviral DNA. Plasmid DNA containing the long terminal repeat sequences flanking a small portion of the gag gene that contains the encapsidation sequence and the genes of interest is transfected into the packaging cell line using standard techniques for DNA transfer and uptake (electroporation, calcium precipitation, etc.). Variants of this approach have been employed to decrease the likelihood of production of replication-competent virus (Jolly, D., Cancer Gene Therapy 1: 51-64 (1994)). The host cell range of the virus is determined by the envelope gene (env) and substitution of env genes with different cell specificities can be employed. Incorporation of appropriate ligands into the envelope protein may also be used for targeting.

Administration of recombinant retroviral vectors may be accomplished by any suitable technique. Such techniques include, for example, ex vivo transduction of patients' cells, direct injection of virus into tissue, and by the administration of the retroviral producer cells. Ex vivo approaches require the isolation and maintenance in tissue culture of the patient's cells. In this context, a high ratio of viral particles to target cells can be achieved and thus improve the transduction efficiency (see, e.g., U.S. Pat. No. 5,399,346, the disclosure of which is incorporated herein by reference). U.S. Pat. No. 4,650,764 contains disclosure relevant to the use of retroviral expression systems and the disclosure of this referenced patent is incorporated herein by reference.

In some cases direct introduction of virus in vivo is necessary or preferred. Retroviruses have been used to treat brain tumors wherein the ability of a retrovirus to infect only dividing cells (tumor cells) may be particularly advantageous.

The administration of a retrovirus producer cell line directly into a brain tumor in a patient has also been proposed (see e.g., Oldfield et al., Hum. Gene Ther. 4: 39-69 (1993)). Such a producer cell would survive within the brain tumor for a period of days, and would secrete retrovirus capable of transducing the surrounding brain tumor.

Pox virus-based systems for expression have been described (Moss and Flexner, Annu. Rev. Immunol 5: 305-324 (1987); Moss, B., In Virology, 1990, pp. 2079-2111). Vaccinia, for example, are large, enveloped DNA viruses that replicate in the cytoplasm of infected cells. Nondividing and dividing cells from many different tissues are infected, and gene expression from a nonintegrated genome is observed. Recombinant virus can be produced by inserting the transgene into a vaccinia-derived plasmid and transfecting this DNA into vaccinia-infected cells where homologous recombination leads to the virus production. A significant disadvantage is that it elicits a host immune response to the 150 to 200 virally encoded proteins making repeated administration problematic.

The herpes simplex virus is a large, double-stranded DNA virus that replicates in the nucleus of infected cells. This virus is adaptable for use in connection with exogenous nucleic acid sequences (see Kennedy and Steiner, Q. J. Med. 86: 697-702 (1993)). Advantages include a broad host cell range, infection of dividing and nondividing cells, and large sequences of foreign DNA can be inserted into the viral genome by homologous recombination. Disadvantages are the difficulty in rendering viral preparations free of replication-competent virus and a potent immune response. Deletion of the viral thymidine kinase gene renders the virus replication-defective in cells with low levels of thymidine kinase. Cells undergoing active cell division (e.g., tumor cells) possess sufficient thymidine kinase activity to allow replication.

A variety of other viruses, including HIV, the minute virus of mice, hepatitis B virus, and influenza virus, have been disclosed as vectors for gene transfer (see Jolly, D., Cancer Gene Therapy 1: 51-64 (1994)).

Nonviral DNA delivery strategies are also applicable. These DNA delivery strategies relate to uncomplexed plasmid DNA, DNA-lipid complexes, DNA-liposome complexes, DNA-protein complexes, DNA-coated gold particles and DNA-coated polylactide coglycolide particles. Purified nucleic acid can be injected directly into tissues and results in transient gene expression for example in muscle tissue, particularly effective in regenerating muscle (Wolff et al., Science 247:1465-1468 (1990)). Davis et al. (Hum. Gene Ther. 4: 733-740 (1993)) has published on direct injection of DNA into mature muscle (skeletal muscle is generally preferred).

Plasmid DNA on gold particles can be “fired” into cells (e.g. epidermis or melanoma) using a gene-gun. DNA is coprecipitated onto the gold particle and then fired using an electric spark or pressurized gas as propellant (Fynan et al., Proc. Natl. Acad. Sci. U.S.A. 90: 11478-11482 (1993)). Electroporation has also been used to enable transfer of DNA into solid tumors using electroporation probes employing multi-needle arrays and pulsed, rotating electric fields (Nishi et al., Cancer Res. 56: 1050-1055 (1996)). High efficiency gene transfer to subcutaneous tumors has been claimed with significant cell transfection enhancement and better distribution characteristics over intra-tumoral injection procedures.

Lipid-mediated transfections are preferred for both in vitro and in vivo transfections (Horton et al., J. Immunology 162: 6378 (1999)). Lipid-DNA complexes are formed by mixing DNA and lipid 1 to 5 minutes before injection, using commercially available lipids such as DMRIE-C reagent.

Liposomes work by surrounding hydrophilic molecules with hydrophobic molecules to facilitate cell entry. Liposomes are unilamellar or multilamellar spheres made from lipids. Lipid composition and manufacturing processes affect liposome structure. Other molecules can be incorporated into the lipid membranes. Liposomes can be anionic or cationic. Nicolau et al. (Proc. Natl. Acad. Sci. U.S.A. 80:1068-1072 (1983)) has published work relating to insulin expression from anionic liposomes injected into rats. Anionic liposomes mainly target the reticuloendothelial cells of the liver, unless otherwise targeted. Molecules can be incorporated into the surface of liposomes to alter their behavior, for example cell-selective delivery (Wu and Wu, J. Biol. Chem. 262: 4429-4432 (1987)).

Feigner et al. (Proc. Nat Acad. Sci. U.S.A. 84: 7413-7417 (1987)) has published work relating to cationic liposomes, demonstrated their binding of nucleic acids by electrostatic interactions and shown cell entry. Intravenous injection of cationic liposomes leads to transgene expression in most organs on injection into the afferent blood supply to the organ. Cationic liposomes can be administered by aerosol to target lung epithelium (Brigham et al., Am. J. Med. Sci. 298: 278-281 (1989)). In vivo studies with cationic liposome transgene delivery have been published (see, e.g., Nabel et al., Rev. Hum. Gene Ther. 5: 79-92 (1994); Hyde et al., Nature 362: 250-255 (1993) and; Conary et al., J. Clin. Invest 93: 1834-1840 (1994)).

Microparticles are being studied as systems for delivery of DNA to phagocytic cells such approaches have been reported by Pangaea Pharmaceuticals. Such a DNA microencapsulation delivery system has been used to effect more efficient transduction of phagocytic cells, such as macrophages, which ingest the microspheres. The microspheres encapsulate plasmid DNA encoding potentially immunogenic peptides which, when expressed, lead to peptide display via MHC molecules on the cell surface which can stimulate immune response against such peptides and protein sequences which contain the same epitopes. This approach is presently aimed towards a potential role in anti-tumor and pathogen vaccine development but may have other possible gene therapy applications.

Natural viral coat proteins which are capable of homogeneous self-assembly into virus-like particles (VLPs) have also been used to package DNA for delivery. The major structural coat protein (VP1) of human polyoma virus can be expressed as a recombinant protein and is able to package plasmid DNA during self-assembly into a VLP. The resulting particles can be subsequently used to transduce various cell lines.

Improvements in DNA vectors have also been made and are likely applicable to many of the non-viral delivery systems. These include the use of supercoiled minicircles (which do not have bacterial origins of replication nor antibiotic resistance genes and thus are potentially safer as they exhibit a high level of biological containment), episomal expression vectors (replicating episomal expression systems where the plasmid amplifies within the nucleus but outside the chromosome and thus avoids genome integration events) and T7 systems (a strictly a cytoplasmic expression vector in which the vector itself expresses phage T7 RNA polymerase and the therapeutic gene is driven from a second T7 promoter, using the polymerase generated by the first promoter). Other, more general improvements to DNA vector technology include use of cis-acting elements to effect high levels of expression, sequences derived from alphoid repeat DNA to supply once-per-cell-cycle replication and nuclear targeting sequences.

As discussed above, the present invention relates to inhibition of Ii in a variety of animal cell types, either in vivo or ex vivo. A broad division among animal cell types, which is relevant to the present discussion, can be made on the basis of the status of MHC Class II molecule expression. This broad division will be introduced briefly here, and revisited within the context of specific therapeutic approaches.

Naturally occurring antigen presenting cells (sometimes referred to as professional antigen presenting cells) participate in the acquired immune response. These cells, which include dendritic cells, macrophages, B lymphocytes and certain other mononuclear cells are MHC Class II molecule-positive. In addition, some cells such as T lymphocytes, do not exhibit MHC Class II molecules in a resting state but may be induced to express MHC Class II molecules upon appropriate activation. Such cells which can be so induced in vivo or in vitro to a function of MHC Class II-restricted presentation of antigenic peptides are included in the category of naturally occurring antigen presenting cells. Cells may be induced to express MHC Class II molecules via co-culturing with autologous serum, IFN-γGM-CSF as described for polymorphonuclear cells (Rasdak, Immunol 101(4): 521-30 (2000)). T-cells may also be induced to express MHC Class II molecules and assume antigen presenting cell functionality when cultured with mitogens and xenogeneic APCs (Patel, J. Immunol. 163(10): 5201-10 (1999)).

As will be discussed in greater detail in following sections, it is possible to introduce into such cells, an expressible nucleic acid sequence encoding an antigenic epitope of interest. When this epitope expression is combined with Ii inhibition, the antigenic epitope of interest is displayed on the surface of the antigen presenting cell in association with MHC Class II molecules.

Naturally occurring antigen presenting cells circulate throughout the body and thorough the peripheral lymphoid tissue. The peripheral lymphoid tissue is organized around the two fluid systems of the body, the blood and the lymph. These two fluid systems are in contact. Lymph is formed by fluid transported from the blood to the spaces within and around tissues. From these extracellular spaces, lymph flows into thin-walled lymphatic vessels, where it is slowly moved to larger central collecting vessels. Ultimately the lymph is returned to veins, where it re-enters the blood. In blood, lymphocytes constitute 20-30 percent of the nucleated cells; in lymph they constitute 99 percent. Antigen presenting cells circulating within these fluid systems pass through the lymph nodes and follicle centers of the spleen. High concentrations of T lymphocytes and B lymphocytes in the lymph nodes of the body and follicle centers of the spleen facilitate cellular interaction and clonal expansion.

Other cells of interest, which typically express little or no MHC Class II molecules, include the vast majority of malignant and virally-infected cells. It is noted, in particular, that some tumors which are usually considered to be MHC Class II-negative, have been reported to express low levels of MHC Class II molecules on some or all of the cells. These include, for example, breast, lung, or colon carcinomas. These cells may express pathology-specific antigens, but given the absence or relatively low abundance of MHC Class II molecules, there is no significant degree of MHC Class II presentation of peptides from such antigens by such cells. In these cells, it is possible both to induce MHC Class II molecule expression as well as to inhibit Ii expression, (Ii expression and MHC Class II expression are co-regulated). This combination intervention results in the display of pathology-related, antigenic epitope-containing peptides on the surface of the cell in association with MHC Class II molecules.

Another class of cells which are of interest is neither malignant, virally infected nor naturally occurring antigen presenting cells. Examples of such cells include fibroblasts, keratinocytes and muscle cells. The cells are MHC Class II molecule-negative and are not classified as naturally occurring antigen presenting cells. Such cells are useful in connection with vaccination methods, either in vivo or ex vivo. Consider, for example, an in vivo context in which muscle cells are targeted for MHC Class II molecules associated antigen presentation. Expressible nucleic acid sequences encoding an antigenic epitope of interest and an inducer of MHC Class II molecules can be injected into muscle tissue. Such sequences are taken up by muscle cells within the tissue and expressed. A percentage of the muscles cells within the area of injection will ultimately express the antigenic epitope of interest, in association with MHC Class II molecules, on the cell surface. Cells competent for stimulation by such a presentation (e.g., helper T cells) will contact presenting cells as the stimulation-competent cells circulate in the lymph. As mentioned above, lymphocytes constitute 99% of the nucleated cells in circulating lymph. Stimulated antigen presenting cells will collaborate with T lymphocytes and B lymphocytes in the lymph nodes of the spleen, where the concentration of cells and other factors facilitate the interaction and magnifies the clonal selection. Antibody produced by secreting B lymphocytes and their mature progeny, the plasma cells, leaves the node in the lymph and is transported to the blood.

The immediately preceding section served to introduce, with limited contextual discussion, cell types of interest for Ii suppression. The discussion which follows will explore these introduced cell types and related methods in greater detail.

Ii suppression therapy is indicated in connection with neoplastic diseases. These include, for example, cancers having a determined primary site, as well as metastatic cancer of unknown primary site. The former class includes breast cancer, malignant tumors of the head and neck, carcinoma of the ovary, testicular cancer and other trophoblastic diseases, skin cancer, and melanoma and other pigmented skin lesions.

Ii suppression therapy is also indicated for certain cells that over-express PAI-1 and have been induced to express MHC Class II molecules. Such cells are found in atherosclerotic plaques in coronary, carotid, renal arteries, veins, and cancer cells. PAI-1 over-expression is associated with tumor invasion, neoangiogenesis and metastasis, as well as myocardial infarction, athersclerosis, restenosis, and thrombembolic disease (U.S. Pat. No. 6,224,865; Gunther, J. Surg. Res. 103(1): 68-78 (2002); Harbeck, J. Clin. Oncol. 20(4): 1000-7 (2002); DeYoung, Circulation 104(16): 1972-1 and (2001); Rerolle, Nephrologie 22(1): 5-13 (2001)). Plasminogen activator inhibitor type 1 (PAI-1) is increased in the arterial walls of patients with diabetes, contributing to the accelerated athersclerosis and plaque progression observed clinically in patients with diabetes (Pandolfi, Arterioscler. Thromb. Vasc. Biol. 21(8): 1378-82 (2001)). PAI-1 activity has been suppressed through the use of specific antibodies, peptidic antagonists, antisense and decoy oligonucleotides (Rerolle, Arterioscler. Thromb. Vasc. Biol. 21(8): 1378-82 (2001)).

Ii suppression therapy is also indicated in connection with infectious diseases. These include viral diseases (DNA and RNA viruses), bacterial diseases (gram-positive and gram-negative), mycobacterial diseases, spirochetal diseases, Rickettsial disease, mycoplasmal and chlamydial diseases, fungal infections, protozoal and helminthic infections and ectoparasitic infections.

With respect to the naturally occurring antigen presenting cells, in vivo and ex vivo applications are included. In the present disclosure, the term “targeting” is sometimes used to describe the directing of an immune response toward an antigenic protein or a particular antigenic epitope within an antigenic protein. This immune response is characterized, in part, by the activation of T immunoregulatory cells, such as T helper cells or T suppressor cells, which may be variably Th1, or Th2, or Th3 cells, depending upon the context of the response. For example a Th1 response is a helper response with respect to development of a CTL response to a tumor antigen, which response leads to killing of tumor cells. A Th1 response to an allergen, however, may be functionally a suppressing response, with respect to immunodeviating the response to the allergen away from a Th2 response, which leads to production of pathogenic IgE antibodies. In addition, the concept of targeting includes, not only the initial portions of the immune response which are stimulated by the presentation of MHC Class II-presented epitopes which are novel or in increased amounts, but also those downstream effector responses which are induced or regulated by the initial actions on T immunoregulatory cells. Thus, for example, targeting includes the CTL-anticancer response or the immunoglobulin anti-viral response which may be initiated by the method of targeting taught herein.

Targeting includes the concept that the immune response is directed to an antigen, whether the antigen either is specified or is not known, nor even identifiable without undue experimentation. For example, targeting may be directed to a cell that may express a large number of antigens each of which may contribute to the generation of an immune response. What particular antigens within a cell participate in the immune response may vary from person to person depending upon the genetic makeup of the individuals. The susceptibility of the immune response to genetic factors has been well described. Consequently, in using the method of targeting for a useful therapeutic or diagnostic purpose, the specific antigenic components of the cell need not and often cannot be specified.

The process of targeting includes processes occurring either in vivo or in vitro. In vivo, for example, the activation of immunoregulatory T cells to antigen presented by an MHC Class II-positive cells which are either tumor cells or dendritic cells may occur in either in a non-tumor location or infiltrating a tumor. The expansion of the effector portion of the immune response likewise may occur either in vivo or in vitro. In the case of in vitro responses, products can be generated which may be reintroduced into the individual, or into another selected individual, to effect a therapeutic response. Examples, of such products include dendritic cell preparations, cytotoxic T cell preparations, and antibodies which might have been produced after cloning B cells from such an in vitro targeted culture, for example after the production of B cell hybridomas.

Toward this end, depending upon the therapeutic product desired for introduction into the individual from which peripheral blood mononuclear cells had been obtained, the original cultures might be fractionated to enrich for a desired cell population, for example, dendritic cells or T lymphocytes. In addition, the culture after the targeting process taught herein has been effected, may be fractionated for a desired cell population, for example, dendritic cells or T lymphocytes. Established methods are available for the fractionation of cells obtained from an individual either immediately after isolation and before the targeting process of this invention, or subsequently after that targeting process has been effected. Furthermore, established procedures are available for the introduction of such products into the individual from which peripheral blood mononuclear cells were originally obtained. To this end, the methods of this invention with respect to targeting are not limited to peripheral blood mononuclear cells, but include all cellular preparations which might be obtained from an individual including mucosal cells from the oropharynx or other regions, cells obtained after bronchial or gastric lavage, cells obtained by biopsy or excision from any organ, such as tumor tissues or normal tissues for example from liver, pancreas, prostate, skeletal muscle, fat, skin.

In all cases, the object is to introduce into a naturally occurring antigen presenting cell an antigenic epitope of interest, which is specific for the pathological condition to be treated, as well as a suppressor of Ii expression. Tumor or virus gene-transfected dendritic cells elicit a strong anti-tumor or anti-virus immune response. Inhibition of Ii protein expression in such antigen gene-transfected dendritic cells will enhance the efficacy of such DNA vaccinations. In the case of both in vivo and ex vivo embodiments involving naturally occurring antigen presenting cells, it is preferable to introduce an expressible nucleic acid sequence encoding the antigenic epitope of interest, and an inhibitor of Ii which may be a reverse gene construct or copolymer such as an antisense or siRNA composition.

The term “expressible nucleic acid sequence” is intended to encompass transcription-competent DNA constructs encoding translation-competent RNA species, as well as translation-competent mRNA species that are transcribed prior to introduction. Those of skill in the art are familiar with the molecular signals required to impart transcriptional and translational competency.

In all embodiments of the invention, it is possible to provide both of these required elements as a single molecular construct (e.g., using a viral vector delivery system having a sufficient capacity to accept nucleic acid encoding both the epitope and the Ii inhibitor). Additional sequences may be included in this single molecular construct, as, for example, when conversion of an MHC Class II molecule-negative cell to an MHC Class II moleule-positive cell is desired. In this case, an expressible nucleic acid sequence encoding a protein that effects the conversion may be included. Such proteins include, for example, CIITA and interferon gamma as discussed hererin. Alternatively, separate expression constructs may be used to carry each element. In the case of separate constructs, delivered in an independent manner, the likelihood of a single antigen presenting cell taking up each of the two constructs is an issue of statistical probability. Furthermore, packaging more than one construct in a single viral particle has the utility of maximizing the therapeutically effective induction of Ii suppression, and when indicated, of MHC Class Ii induction and/or induction of the synthesis of a desired protein antigen, relative to the synthesis of viral proteins to which is generated an immune response which is deleterious. Such an anti-viral immune response can for example limit the frequency with which such therapeutic interventions are possible.

Resultingly, a method for displaying an antigenic epitope of interest on the surface of an MHC Class II molecule-positive cell in which Ii protein expression is suppressed may comprise a) providing a cell which is either MHC Class II molecule-positive or is induced to express MHC Class II molecules on its cell surface and further wherein the cell expresses Ii; and introducing into the cell of step a) an antigenic epitope of interest and an inhibitor of Ii. The inhibitor of Ii may be any inhibitor of Ii, and may be a reverse gene construct or copolymer, such as an siRNA or antisense composition, of the present invention. The antigenic epitope of interest may be introduced prior to, subsequent to, or concurrent with the introduction of the inhbitor of Ii. In this method where conversion of an MHC Class II molecule-negative cell to an MHC Class II molecule-positive cell is desired, an expressible nucleic acid sequence encoding a protein that effects the conversion may be introduced at the time the inhibitor of Ii and/or antigenic epitope of interest, although it is not a strict requirement.

Introduction by non-viral delivery systems requires specific consideration as well. Using non-viral delivery systems uncomplexed DNA, DNA-liposome complexes, DNA-protein complexes and DNA-coated gold particles can be delivered into cells. Each of these methods offers advantages and disadvantages which control selection for specific pathologies. The use of complexed DNA (e.g., DNA-liposome complexes, DNA-protein complexes, DNA-coated gold particles, and microencapsulation in polylactide cogylcolide particles) would tend to ensure delivery to a single cell, both the epitope encoding nucleic acid sequence and the nucleic acid sequence encoding the inhibitor of Ii expression. Even if encoded by distinct molecular species, both species would tend to be delivered to a single cell because they are “packaged” (e.g., either encapsulated in a liposome, or coated onto a gold particle).

DNA-coated gold particles are commonly delivered by a ballistic method using the so-called “gene gun” technology. Using this technique, gold particles can be fired into the skin or muscle tissue and used to penetrate cells. Penetrated cells have been shown to express nucleic acid sequences introduced in this manner. Dendritic cells are naturally occurring antigen presenting cells which are effectively transfected using this technique. Such expression constructs, when introduced into a single dendritic cell, for example, will result in the display of the antigenic epitope of interest on the surface of the antigen presenting cell in association with MHC Class II molecules. The display of the epitope/MHC Class II molecule complex on the surface of the antigen presenting cell will stimulate additional immune cells providing a heightened immune response.

Alternatively, when addressing a pathological condition having a defined anatomical location (e.g., primary tumors or some metastases of neoplastic diseases), direct injection into the defined anatomical site may be indicated. Such sites will tend to be enriched in antigen presenting cells such as dendritic cells. A tumor is an example of such a local site of introduction. A means for accomplishing the introduction of the relevant expressible nucleic acid constructs into cells is appropriate. When introducing such constructs into a localized tumor site, it is preferable to include an additional expressible nucleic acid sequence encoding a protein which stimulates expression of MHC Class II molecules. The inclusion of this third component is intended for the tumor cells themselves. If the construct which inhibits Ii expression, and the expressible inducer of MHC Class II molecules production are taken up by a cell exhibiting a pathology (e.g., a tumor cell), the cell will display pathology-specific epitopes on its cell surface in association with MHC Class II molecules. These cells also will stimulate T helper cells and B lymphocytes. Thus, the direct injection of these three expressible elements in connection with a therapy directed toward a localized pathology can be viewed as a combination therapy in which normal antigen presenting cells and MHC Class II molecule-negative cells exhibiting the pathology are targeted.

In addition to the use of an expressible nucleic acid sequence to induce MHC Class II molecule production, those skilled in the art will recognize the applicability of the process of nuclear transfer in this and related contexts (Wolf, Arch. Med. Res. 32(6): 609-13 (2001); Wakayama, Science 292(5517): 740-3 (2001)). In addition, an antigen presenting cell may be derived from a somatic cell that has been induced to de-differentiate thereby expressing onco-embryonic antigens (Rohrer, J. Immunol. 162(11): 6880-92 (1999)). Such cells may be used to induce immune attack on antigenic epitopes of interest. Fully differentiated cells in vivo may be induced to de-differentiate to premature forms to effect organ regeneration (Abbate, Am. J. Physiol. 277(3 Pt 2): F454-63 (1999)). These cells may also function as antigen presenting cells in order to stimulate an immunological attack on aberrant cells (Fu, Lancet 358(9287): 1067-8 (2001)).

In another embodiment of the present invention in which antigen presenting cells are targeted in vivo, normal tissue is stimulated by subcutaneous injection of a cytokine (e.g., GM-CSF). This subcutaneous “priming” attracts dendritic cells to the area. The priming injection is followed by the injection of expressible nucleic acid sequences encoding an antigenic epitope of interest, as well as an inhibitor of Ii synthesis (e.g., an siRNA). Neoplastic cells are producers of pathology-specific peptides, but generally do not present them on their surfaces in association with MHC Class II molecules. In such cells, it is possible to both induce MHC Class II molecules expression as well as inhibit Ii expression. For example, the expression of MHC Class II molecules can be induced by introducing into the MHC Class II molecules-negative cell, a cDNA coding for a protein which stimulates MHC Class II molecules production. Such proteins include, for example, CIITA or interferon gamma. This combination intervention results in the display of pathology-specific, antigenic epitope-containing peptides on the surface of the cell in association with MHC Class II molecules. As discussed previously, the introduction of expressible nucleic acid sequences is the preferred method of accomplishing these goals.

As discussed above, direct injection is indicated where the pathology presents a defined primary locus (such as a tumor). Optionally, an expressible nucleic acid sequence encoding an antigenic epitope of interest may be included in the injected material to target antigen presenting cells in the area. Again, the goal for delivery to the antigen presenting cell is the Ii suppressor and the antigenic epitope of interest. For the pathological cells, the delivery goal is the Ii suppressor and the MHC Class II molecules inducer.

Specific protocols followed in connection with an intratumor injection are based, for example, on established therapeutic protocols involving intratumoral injection of cytokine encoding nucleic acid sequences, or cytokines. Such protocols are described, for example, in a number of publications including: Schultz, J., Cancer Gene Ther. 7(12): 1557-650 (2000); Mastrangelo, M. J., Cancer Gene Ther. 6(5): 409-22 (1999); Toda, M., Mol. Ther. 2(4): 324-9 (2000); Fujii, S., Cancer Gene Ther. 7(9): 1220-30 (2000); Narvaiza, I., J. Immunol. 164(6): 3112-22 (2000); Wright, P., Cancer Biother. Radiopharm. 14(1): 49-57 (1999); Cancer Res. 58(8): 1677-83 (1998); Staba, M. J., Gene Ther. 5(3): 292-300 (1998); U.S. Pat. No. 5,833,975; U.S. Pat. No. 6,265,189 B1; Griffith, T. S., J. Natl. Cancer Inst 93(13): 998-1007 (2001); Siemens, D. R., J. Natl. Cancer Inst. 92(5): 403-12 (2000); Sacco, M., Gene Ther. 6(11): 1893-7 (1999); Cao, X., J. Exp. Clin. Cancer Res. 18(2): 191-2000 (1999); Wright, P., Cancer Gene Ther. 5(6): 371-379 (1.998); Nasu, Y., Gene Ther. 6(3): 338-49 (1999); U.S. Pat. No. 6,034,072; Lotze, M. T., Cancer J. Sci. Am. 6 Suppl 1: S61-66 (2000); Schmitz, V., Hepatology 34(1): 72-81 (2001); Wang, Q., Gene Ther. 8(7): 542-50 (2001); Dow, S. W., J. Clin. Invest 101(11): 2406-14 (1998); Kagawa, S., Cancer Res. 61(8): 3330-8 (2001); Addison, C. L., Gene Ther. 5(10): 1409-9 (1998); Lohr, F., Cancer Res. 61(8): 3281-4 (2001); Yamashita, Y. I., Cancer Res. 61(3): 1005-12 (2001); Kirk, C. J., Cancer Res. 61(5): 2062-70 (2001); Hum. Gene Ther. 12(5): 489-502 (2001); Putzer, B. M., J. Natl. Cancer Inst. 93(6): 472-9 (2001); Mendiratta, S. K., Hum. Gen. Ther. 11(13): 1851-62 (2000); International Publication No. WO 99/47678; Natsume, A., J. Neurooncology 47(2): 117-24 (2000); Peplinski, G. R., Surgery 118(2): 185-90 (1995); deWilt, J. H., Hum. Gene Ther. 12(5): 489-502 (2001); Emtage, P. C., Hum. Gene Ther. 10(5): 697-709 (1999); Clin. Cancer Res. 3(12 Pt 2): 2623-9 (1997); Chen, S. H., Mol. Ther. 2(1): 39-46 (2000); Putzer, B. M., Proc. Natl. Acad. Sci. USA 94(20):10889-94 (1997); Walther, W., Cancer Gene Ther. 7(6): 893-900 (2000); Fushimi, T., J. Clin. Invest. 105(10): 1383-93 (2000); Xiang, J., Cancer Gene Ther. 7(7): 1023-33 (2000).

With respect to ex vivo applications, tumor cells are isolated from the individual and an ex vivo culture is established. Such cultures can be established from an unselected population of malignant cells obtained from the individual, with or without separation from accompanying normal cells, or cells can be obtained as cell lines or clones from such cell lines. Alternatively, such cells are obtained from established malignant cell lines of unrelated patients or as explants of fresh malignant tissue (e.g., colon or ovarian carcinoma).

Ii suppressor and MHC Class II molecules inducers are introduced into the cultured cells, resulting in the desired MHC Class II molecules-associated presentation of tumor-specific or tumor-related antigenic epitopes. Inducers of MHC Class II molecules expression are well known in the art and include, for example, MHC Class II molecule transacting factor (CIITA), interferon gamma gene and interferon gamma cytokine. Cells treated in this manner are rendered replication incompetent (e.g., by irradiation or fixation), and used in a conventional immunization protocol (e.g., subcutaneous, intravenous, intraperitoneal or intramuscular immunization). In addition to whole cell formulations, other derivative thereof may be used in the immunization formulation.

Although a majority of the relevant tumor cells are MHC Class II molecule and Ii-negative, it is well-known that some tumors (for example, certain lymphomas, melanomas and adenocarcinomas, affecting, for example, breast, lung and colon) are MHC Class II molecule-positive and Ii-positive. In this subset which express MHC Class II molecules, the introduction of only an Ii suppressor may be adequate to achieve the desired immune stimulation. It will be recognized that the inclusion of an MHC Class II molecule inducer in such cells may serve to enhance the desired stimulation by increasing the likelihood of T helper cell interaction with MHC Class II molecules-associated antigen.

Another class of cells which are of interest is neither malignant, virally infected nor naturally occurring antigen presenting cells. Expressible nucleic acid sequences are delivered to the interstitial space of tissues of an individual. Such tissues include, for example, muscle, skin, brain, lung, liver, spleen, bone marrow, thymus, heart, lymph, blood, bone, cartilage, pancreas, kidney, gall bladder, stomach, intestine, testis, ovary, uterus, rectum, nervous system, eye, gland and connective tissue. Interstitial space of tissues comprises the intercellular, fluid, mucopolysaccharide matrix among the reticular fibers of organ tissues, elastic fibers in the walls of vessels or chambers, collagen fibers of fibrous tissues, or that same matrix within connective tissue ensheathing muscle cells or in the lacunae of bone. It is similarly the space occupied by the plasma of the circulation and the lymph fluid of the lymphatic channels.

It has been reported that in vivo muscle cells are particularly competent in their ability to take up and express an expressible nucleic acid sequence (see, for example, U.S. Pat. No. 6,214,804, the disclosure of which is incorporated herein by reference). This delivery advantage may be due to the singular tissue architecture of muscle, comprising multinucleated cells, sarcoplasmic reticulum and transverse tubular system. Expressible nucleic acid sequences may enter the muscle through the transverse tubular system, which contains extracellular fluid and extends deep into the muscle cell. It is also possible that such expressible nucleic acid sequences enter damaged muscle cells which then recover.

Muscle is also advantageously used as a site for the delivery of an expressible nucleic acid sequence in therapeutic applications because animals have a proportionately large muscle mass which is conveniently accessed by direct injection through the skin. For this reason, a comparatively large dose of expressible nucleic acid sequence can be deposited in muscle by multiple and repetitive injections. Therapy can be extended over long periods of time and are safely and easily performed without special skill and equipment. Tissues other than those of muscle, and being characterized by a less efficient uptake and or expression of an expressible nucleic acid sequence, may also be used as injection sites.

In connection with the present invention, it is desirable to inhibit Ii synthesis in the target cell, and also to express an antigenic epitope of interest (the antigenic epitope being specifically associated with a pathological condition to be treated). As is known in the art, an effective dosage of expressible nucleic acid sequence will generally fall within the a range of from about 0.05 micrograms/kg body weight, to about 50 mg/kg body weight (commonly about 0.005 mg/kg to about 5 mg/kg). It will be recognized that effective dosages can vary depending upon a number of relevant factors.

Another method for generating a cell which produces a pathology-associated antigen of the type described above, and displays it on a cell surface in association with MHC Class II molecules, is the cell fusion methodology. More specifically, it is a matter of routine experimentation to produce a fusion of a cell which naturally produces MHC Class II molecules (e.g., a naturally occurring antigen presenting cells such as a dendritic cell), with a cell exhibiting a pathology of interest (e.g., a tumor cell). In such a fusion cell, tumor-specific antigen will be displayed in association with MHC Class II molecules on the surface of the fusion cell. In most cases, the product is a fusion of a class of naturally occurring antigen presenting cells, such as dendritic cells, macrophages, B lymphocytes, or certain multipotent cells, and cells which express the antigenic epitopes of interest. Such cells expressing antigenic epitopes of interest include, for example, malignant cells, virally infected cells or transformed cells, cells relevant to induction of an autoimmune response, and cells regulating the autoimmune response. The latter class includes cells which exert their influence through anti-idiotypic network mechanisms (e.g., expressing the T cell receptor of pathogenic relevance in rheumatoid arthritis). Cell fusions of this type are produced ex vivo. Ii suppression and vaccination are carried out as described elsewhere in this disclosure.

It will be recognized by one of skill in the art that methods of the present invention may be combined with a cytokine therapy (i.e., the introduction of cytokine encoding nucleic acid sequences, or cytokines themselves), into the cells to be treated, or their local environment. Other immune co-stimulatory molecules may be used as well (Akiyama, Y., Gene Ther. 7(24): 2113-21 (2000); Miller, P. W., Hum. Gene Ther. 11(1): 53-65 (2000); J. Neurosurg. 94(2): 287-292 (2001); Jantscheff, P., Cancer Immunol. Immunother. 48(6): 321-30 (1999); Kikuchi, T., Blood 96(1): 91-9 (2000); Melero, I., Gene Ther. 7(14): 1167-70 (2000); Lei, H., Zhongua Zhong Liu Za Zhi 20(3): 174-7 (1998)).

As previously mentioned, one of skill in the art could, without undue experimentation, identify pathology-specific antigen for which MHC Class II molecule-associated display would provide an enhanced immune response. Again, in all cases, Ii inhibition would be required in order to effect the display of such antigen in association with MHC Class II molecules. The following list is intended to be a non-limiting, non-exhaustive listing of examples of antigen falling within this class: HIV gp120 (Barouch et al., J. Immunol. 15; 168: 562-8 (2002)); HIV gag (Singh et al., Vaccine 20: 594-602 (2001)); Influenza M1 and M2 (Okuda et al., Vaccine 19: 3681-91 (2001)); Hepatitis B surface antigen and core antigen (Musacchio et al., Biochem. Biophys. Res. Commun. 282: 442-6 (2001)); Human telomerase reverse transcriptase (hTERT) (Heiser et al., Cancer Res. 61: 3388-93 (2001)); Gp75TRP-1 (Bowne, Cytokines Cell Mol. Ther. 5: 217-25 (1999)); TRP-2 and gp100 (Xiang, Proc. Natl. Acad. Sci. USA 97: 5492-7 (2000)); PSA (Kim, Oncogene 20(33): 4497-506 (2001)); CEA (von Mehren et al., Clin. Cancer Res. 7: 1181-91 (2001)); Erb2/Neu (Pilon et al., Immunol. 167: 3201-6 (2001), and Tuting, Gene Ther. 6: 629-36 (1999)).

In another aspect this invention addresses genetic recombination in infectious viruses in a manner to promote the immune response to such constructs when administered as prophylactic or therapeutic vaccines. A wide range of viral vaccines is suitable to these methods of genetic modification, although the genetic recombinants, and methods for their construction and use, differ according to the virus of interest. To this end, specific approaches to design and use of recombinant DNA viruses, with vaccinia as a prototypic example, and of RNA viruses, with influenza virus as a prototypic example, are considered.

There are two formats for vaccine viruses of either the DNA or RNA types. One contains an Ii-RGC or a construct for expression of an siRNA leading to suppression of Ii protein expression in the infected cell, and the second has both a) an Ii-RGC or a construct for expression of an siRNA leading to suppression of Ii protein expression in the infected cell and b) a gene construct leading to expression of MHC class II molecules, e.g., genes for CIITA or interferon-γ. In the case of a DNA virus, such as vaccinia, the genes are under the control of classical mammalian promoters such as CMV, RSV, Ubc, EF-1α, and U6. In the case of RNA viruses, such as influenza, translation from the RNA of the inserted constructs are expressed by the influenza viral enzyme mediating RNA transcription and translation mechanisms. The first type of virus, with only the capacity to suppress expression of Ii protein in the infected cell, is targeted for cell types which already endogenously express Ii protein and MHC class II molecules. Such cell types include Langerhans cells of the skin, other dendritic cells in skin or in mucosal surfaces of the respiratory tract or gut, or which might have been mobilized from bone marrow, or obtained from bone marrow or spleens, macrophages of the peripheral blood or other bodily fluids such as exudative or transudative fluids arising or induced in abdominal, pleural, pericardial of other bodily cavities. Additional cell types include B cells, or B lineage leukemias and lymphomas, and cells which by activation have come to express MHC class II molecules and Ii protein, such as some subsets of T cells and transformed malignant or normal cells. The second type of virus construct (type b above) can transfect and regulate Ii expression in all of the cell types listed above for infection by type a) viruses, but in addition can transfect keratinocytes, or muscle cells, or other cells which do not normally express MHC class II molecules and Ii protein, but which can be induced under the influence of the virus-incorporated genetic sequence leading to induction of those molecules, e.g., by CIITA or interferon-γ.

The construction of examples of these two classes of DNA or RNA viruses can be achieved with standard molecular biological techniques. The cDNA encoding CIITA and Ii-specific siRNA can be introduced using standard molecular cloning methods into plasmids encoding vaccinia, canarypox, or other DNA viruses (Panicali D. Proc Natl Acad Sci USA. 1982; 16:4927-31). Intact vaccinia viral DNA as well as CIITA and Ii-specific siRNA expression cassettes can be cloned into a vector flanked by viral sequences. Homologous recombination between the cloned CIITA and Ii-specific siRNA expression cassettes can occur and novel viruses can be selected under the appropriate conditions (Panicali D. Proc Natl Acad Sci USA. 1982; 16:4927-31; Marti W R. Cell Immunol. 1997:179:146-52; Bertley F M N. J. Immunol. 2004; 172:3745-57). Recombinant RNA viruses can be similarly constructed using plasmids encoding viral cDNAs. A plasmid-based reverse genetics system for Influenza A virus has been developed (Pleschka S. J Virol 1996; 70:4188-92). This system uses plasmids containing a truncated human polymerase I promoter to express viral RNA. CIITA and Ii-specific siRNA expression cassettes can be cloned into a plasmid encoding the influenza HA or NA gene. Plasmids encoding all 8 segments of the viral genome can be cotransfected into tissue cultured cells to recover infectious recombinant viruses that can be used for vaccination purposes. Alternatively, a recombinant plasmid encoding CIITA and Ii-specific siRNA can be transfected into a cell line infected with an influenza helper virus. Using a selection method, viruses containing the genetically engineered transfectant virus can be isolated (Palese P. J. Virol. 1996; 93:11354-8). Design and preparation of these various constructs, and their applications as vaccines, can be executed with the materials and methods of the following US patents. U.S. Pat. No. 5,976,552, U.S. Pat. No. 5,292,506, U.S. Pat. No. 4,826,687, U.S. Pat. No. 6,740,325, U.S. Pat. No. 6,651,655, U.S. Pat. No. 5,948,410, U.S. Pat. No. 5,824,536, U.S. Pat. No. 4,029,763, U.S. Pat. No. 4,009,258, U.S. Pat. No. 668,463, U.S. Pat. No. 667,611, U.S. Pat. No. 6,623,962, and U.S. Pat. No. 6,506,559. The recombinant viruses expressing CIITA and Ii-specific siRNA will be assayed for the ability to enhance MHC Class II responses using in vitro and in vivo models.

### EXEMPLIFICATION

### Example 1

Construction of an Adenoviral Vector Containing the CIITA cDNA.

The initial goal of this experiment was to construct an adenoviral vector for efficient induction of MHC class II molecules in MHC class II molecule negative cells (e.g., MC-38 and Renca). The CIITA gene construct, including a CMV promoter and poly A tail, was excised from a CIITA-containing pCEP4 vector (obtained from Dr. L. Glimcher) using Sal1. This fragment was ligated into pBluescript to create pBlue/CIITA. pBlue/CIITA was then digested with EcoRV and XhoI to release a DNA fragment including the CMV promoter, CIITA cDNA and poly A signal, which was ligated into pQBI/BN (Quantum, Montreal, Canada) to create pQBI/BN/CIITA.

This vector was co-transfected into 293A adenoviral packaging cells with Cla1 digested adenoviral DNA (the left arm of the virus was deleted to reduce background) according to the manufacturer's instruction. Three weeks after co-transfection, resulting plaques were screened by PCR using two DNA primers located at −7 to +12 and +751 to +769 of the CIITA cDNA to ensure the presence of the CIITA gene. One clone was used to test induction of MHC class II molecules in two murine tumor cell lines: MC-38 colon adenocarcinoma and Renca renal cell adenocarcinoma. Time-course for induction of MHC Class II molecules was assayed in these cell lines after infection with the adeno/CIITA recombinant adenoviral vector. An otherwise identical adenovirus vector lacking the CIITA insert was used as a control. It was determined that MHC class II molecules are strongly induced at 48-72 hours after infection in >95% cells.

### Example 2

Generation of the MHC Class II+/Ii− Phenotype by Infection with Adeno/CIITA Plus Treatment with Ii Antisense Oligonucleotides.

This example demonstrates the generation of cells expressing the MHC class II+/Ii− phenotype by infection of cells with adeno/CIITA and inhibition of Ii expression by defined Ii antisense oligonucleotides. The Ii antisense oligonucleotide had been previously demonstrated to be effective (Qiu et al., Cancer Immunol. Immunother. 48: 499-506 (1999)). Control experiments included: a) no treatment; b) adeno/CIITA construct alone; c) adeno/CIITA construct together with sense control oligonucleotide; and d) adeno/CIITA construct together with four-nucleotide mismatched control antisense oligonucleotide. Briefly, 1.5×10⁶ MC-38 cells were seeded into 25 cm² flasks 24 hr before electroporation with oligos and infected in 5 ml total volume media containing 1.5 ml virus stock solution (1.26×10⁶ PFU/ml) for 48 hr. After the first 24 hr of infection, 10 ml of fresh medium was added and cells were incubated for another 24 hr. The cells were then trypsinized and washed prior to electroporation with either antisense, sense or mismatched oligonucleotides. The conditions for electroporation were as follows: 3-5×10⁶ cells were added to an electroporation cuvette in 0.5 ml RPMI 1640 containing 50 μM oligonucleotides. The cells were incubated on ice for 10 min and subjected to 200 volts/1250 μF using a BTX 600 electroporator. The cuvettes were then incubated on ice for another 10 min after which the cells were washed once, seeded into a fresh 25 cm² flask and incubated for 24 hr. At this time the cells were trypsinized and analyzed by flow cytometry following staining for MHC class II molecule and Ii protein as previously described (Qiu et al., Cancer Immunol. Immunother. 48: 499-506 (1999)).

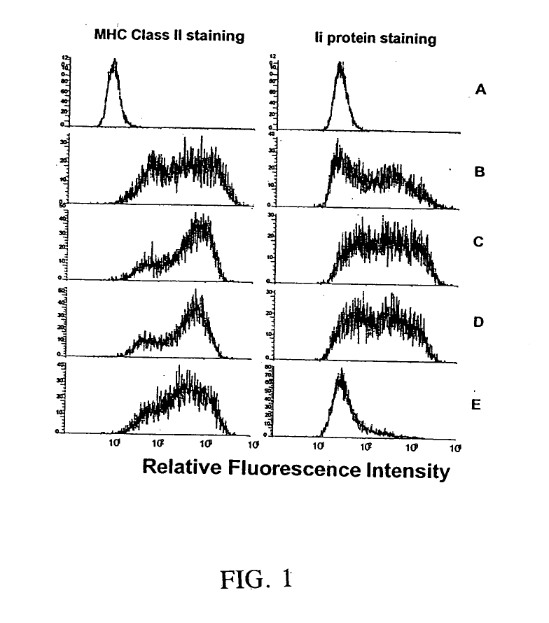

In a typical experiment, shown in FIG. 1, cells that were Ii antisense-treated and adeno/CIITA-infected showed good selective inhibition of Ii with little or no effect on MHC class II molecule expression. The control oligonucleotide-treated cells (i.e., using mismatch or sense sequences) showed no inhibition of Ii and had comparable MHC class II molecule expression relative to adeno/CIITA infected cells.

In anticipation of animal studies and the need to generate MHC Class II.sup.+/Ii.sup.− cells in larger quantities, the above studies were repeated in a scaled-up system. 5×10⁶ MC-38 cells were seeded into a 75 cm² flask 18 to 24 hr prior to infection. The cells were infected with 5 ml of viral stock solution (1.26×10⁶ PFU/ml) for 90 min and 20 ml of fresh medium was added. The cells were then incubated for 48 hours and subjected to electroporation to deliver oligonucleotides (50 μM) as described above. The cells were then pooled and incubated in a fresh 75 cm² flask for another 24 hr, after which the media was changed and the cells incubated for an additional 3 hr. The cells were then analyzed for expression of MHC class II molecules and Ii proteins and for the immunization of mice. Sequence specific inhibition of Ii protein expression was obtained only in cells infected with adeno/CIITA and treated with Ii antisense, as observed in previous experiments.

### Example 3

Tumor Protection by MHC Class II+/Ii− Tumor Vaccine.

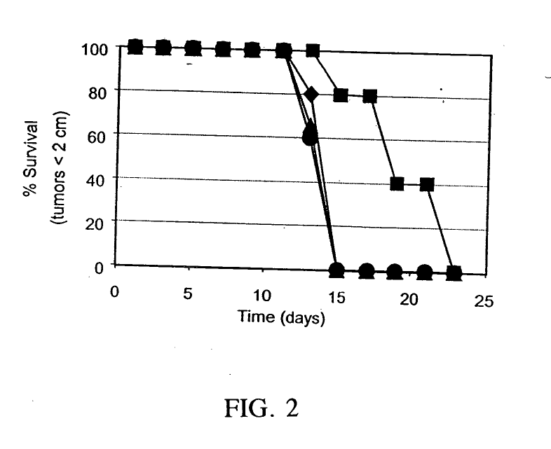

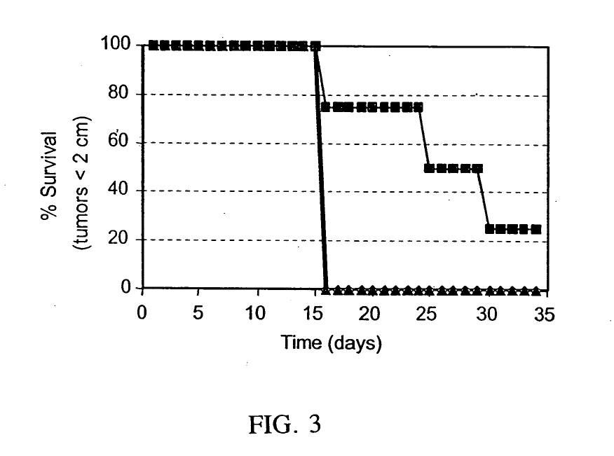

For these studies, MC-38 tumor vaccine cells were prepared as described above and used to inoculate 6-7 week old, female C57BL/6 mice (Jackson Labs). Specifically, MC-38 cells were infected with adeno/CIITA as described, divided into four groups and treated by electroporation with: a) nothing; b) 50 μM Ii antisense oligonucleotide; c) 50 μM mismatch control oligonucleotide; or d) 50 μM sense control oligonucleotide, and seeded into flasks. After 24 hr, fresh media was added and cells were incubated for an additional 3 hr. Cells were then trypsinized, lethally irradiated with 50 Gy (Cesium source) and 1.2×10⁶ cells/mouse were inoculated into mice. Five weeks later, mice were challenged with 5×10⁵ parental MC-38 cells and monitored for appearance of tumors. As shown in FIG. 2, inoculation with Ii antisense treated, adeno/CIITA infected MC-38 cells provided better protection against tumor growth relative to all other control groups. These data are consistent with our previous studies using MC-38 cells stably transfected with CIITA and treated with Ii antisense (FIG. 3). As can be seen, the level of protection using either stably CIITA-transfected MC-38 or transient adeno/CIITA-infected cells treated with Ii antisense gives a comparable level of protection.

### Example 4

Tumor Protection by MHC Class II+/Ii− Tumor Vaccine and GM-CSF.

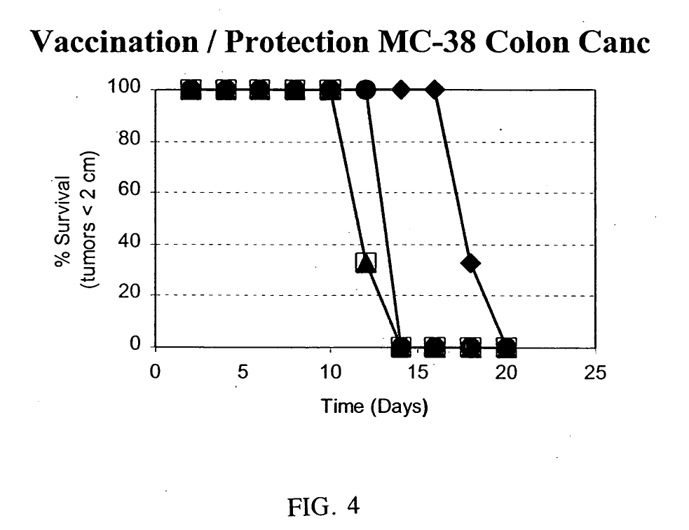

In another set of animal studies, the role of Ii-inhibited MC-38 vaccine cells together with GM-CSF treatment on the subsequent growth of parental MC-38 cells was investigated. For these studies, mice were injected with 18 μg of GM-CSF (R&D system, Minneapolis, Minn.) s.c. in the right rear leg one day before MC-38 cell immunization to attract dendritic cells. Also, the number of MC-38 cells used to immunize mice was only 3×10⁵ cells/mouse, 4 times lower than used in previous experiments. As shown in FIG. 4, GM-CSF enhances the protective effect elicited by class II+/Ii− MC-38 cells. Mice inoculated with only 3×10⁵ class II+/Ii− cells similarly inhibited parental cells growth as it was induced by 1.2×10⁶ of class II+/Ii− MC-38 cells in the absence of GM-CSF. In previous studies, it was shown that MHC induced by IFN-γ offered much stronger induction of anti-tumor immune response than by CIITA (Qiu et al., Cancer Immunol. Immunother. 48: 499-506 (1999)).

These studies indicated that synergistic effect between cytokines and Ii inhibition is feasible. Additional studies are planned to combine the use of GM-CSF and IFN-γ with Ii antisense strategy and to optimize and amplify this immunization protocol.

### Example 5

Construction of an Adenoviral Vector Containing the IFN-γ cDNA.

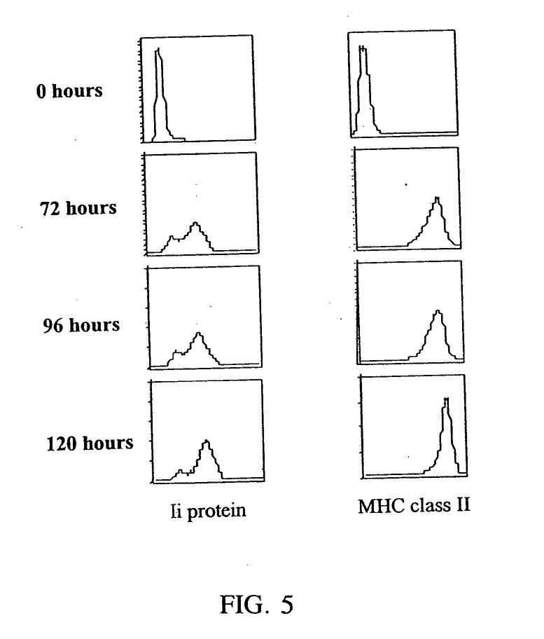

IFN-γ plays an important role in regulating the direction of the immune response and it induces MHC class II molecule and Ii in a variety of tissue and cells including some malignant cells. An MHC class II+/Ii− tumor vaccine created by transfection with an IFN-γ construct and Ii inhibition by antisense oligonucleotides has increased immunogenecity relative to an otherwise identical tumor vaccine in which MHC Class II molecule expression is induced by CIITA transfection (Qiu et al., Cancer Immunol. Immunother. 48: 499-506 (1999)). Expressible murine IFN-γ sequences have been cloned into adenovirus. Expression of both MHC class II molecules and Ii protein were induced following infection of adeno/IFN-γ at very low concentrations (see FIG. 5) (even at an MOI of 1, data not shown). To create the adeno/IFN-γ construct, murine IFN-γ cDNA (Chen et al., J. Immunol. 151: 244-55 (1993)) was amplified by PCR with two specific oligonucleotides complementary to the regions containing the start and stop codons on IFN-γ cDNA. The IFN-γ fragment was cloned into pCDNA(3+) plasmid by using specifically designed endonuclease digestion sites and confirmed by sequencing. The CMV promoter, IFN-γ, and poly A signal was further PCR amplified with appropriate oligonucleotides and then cloned into pQBI/Ad/BN using the appropriate restriction sites. The generation of adeno/IFN-γ recombinant virus was performed by the same procedures described in Example 1.

### Example 6

Construction of an Adenoviral Vector Containing the Ii-RGCs.

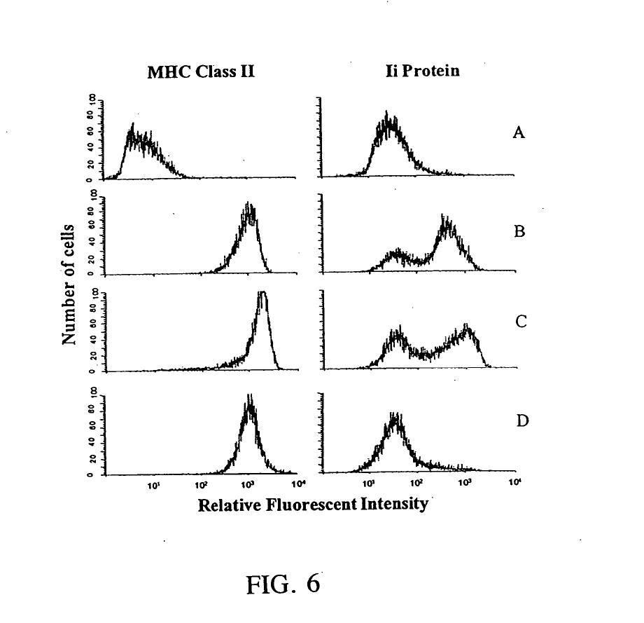

Several Ii reverse gene constructs were cloned in RSV.5 and pcDNA(3+) expression vectors. A subset of the constructs was shown to have the ability to inhibit Ii in MHC class II molecule-positive cells (A20) using classical transfection methods (e.g., lipofectin). While it has been shown that Ii antisense oligonucleotides are also effective, they require electroporation or other methods with significant associated toxicity. Also, no more than 30-70% of cells treated with oligonucleotides demonstrate significant inhibition of Ii expression. In contrast (and as shown using the adeno/CIITA construct), the use of adenoviral vectors for gene delivery results in nearly 100% delivery to all cells, desired phenotypic changes and virtually no toxicity. Several Ii-RGCs were cloned into adenovirus for better induction of MHC class II+/Ii− phenotype. To create the recombinant adenovirus containing Ii-RGCs, the expression cassette consisting of RSV (or CMV) promoter, Ii reverse gene fragment and poly A signal was amplified by PCR and cloned into the pQBI/BN vector using Not1 and Xho1 or other proper restriction enzyme sites to create pQBI/BN/Ii-RGC. Final construction of the adeno/Ii-RGCs was accomplished by the same procedures described in Example 1. In an experiment of induction of MHC class II+/Ii− phenotype, it was observed that when the concentration of adeno/Ii-RGC was increased to about 4 times that of adeno/CIITA, Ii was inhibited in >95% of cells while expression of MHC class II molecules was almost not effected (see FIG. 6).

### Example 7

Construction of an Adenoviral Vector Containing the IFN-γ and Ii-RGCs.

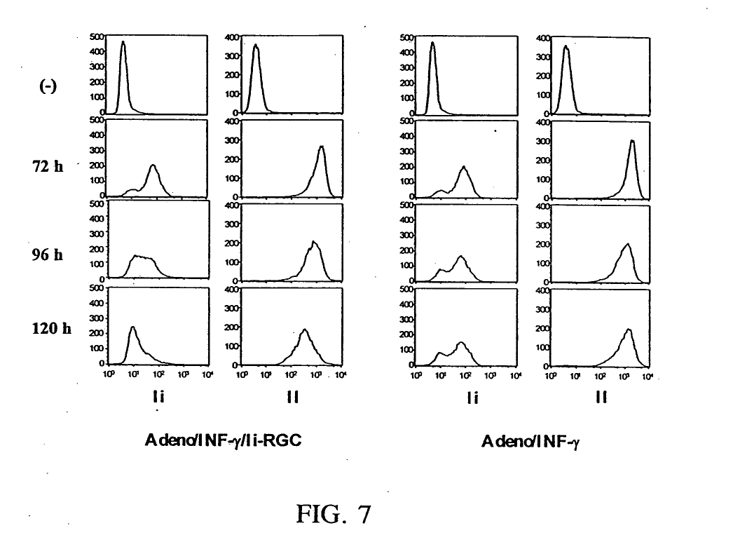

To simplify infection, adeno/IFN-γ/Ii-RGC constructs have been generated. The promoter, Ii-RGC fragment, and poly A signal were amplified by PCR with appropriate oligonucleotides and cloned into pQBI/Ad/BN/IFN-γ to create the pQBI/Ad/BN/IFN-γ/Ii-RGC, which was subsequently used to generate adeno/IFN-γ/Ii-RGCs were made. It was observed, in the MHC class II+/Ii− phenotype induction experiment by infection with adeno/IFN-γ/Ii-RGCs, that the MHC class II+/Ii− phenotype was generated in MC/38 cells by infection with one of the pQBI/Ad/BN/IFN-γ/Ii-RGC constructs (adeno/IFN-γ/Ii-RGC(−92, +9-7)) 96 hours after infection (see FIG. 7).

### Example 8

Construction of an Adenoviral Vector Containing Multiple Ii-RGCs.

In order to maximize the efficacy of the Ii-RGCs, several Ii-RGCs were cloned into one adenoviral vector. PCR amplification and other appropriate molecular biological methods were used to generate the pQBI/Ad/BN constructs containing different combinations of Ii-RGCs. Examples of such constructs included the set shown below. The nucleotide sequences of murine Ii inserts (−92, +97), (+32, +136), (+314, +458) are presented in the Sequence Listing as SEQ ID NOS 1, 2 and 3, respectively.
adeno/(−92, +97)(+31 4, +458)
adeno/(−92, +97)(+314, +458)×2
adeno/(−92, +97)(+31 4, +458)×3
adeno/(−92, +97)(+32, +136)
adeno/(−92, +97)(+32, +136)(+314, +458)

Some of the Ii-RGCs were also cloned with IFN-γ, including the set shown below.
adeno/CIITA/IFN-γ
adeno/CIITA/IFN-γ/(−92, +97)
adeno/IFN-γ/(−92, +97)
adeno/IFN-γ/(−92, +97)(+314, +458)
adeno/IFN-γ/(−92, +97)(+32, +136)(+314, +458)

In a subsequent effort to maximize the effect of Ii-RGCs plasmid containing multiple copies of Ii-RGCs were generated, each being driven by different promoters. These plasmids are described below.
pQBI/Ad/BN//Ii-RGC(−92, 97/−92, 97). The promoters are RSV, EF-1a, respectively.
pQBI/Ad/BN//Ii-RGC(−92, 97/−92, 97/−92, 97). The promoters are RSV, EF-1a, UbC, respectively.
pQBI/Ad/BN/Ii-RGC(−92, 97/32, 136/314, 459). The promoters are RSV, EF-1a, UbC, respectively.
pQBIAd/BN/CIITA/Ii-RGC(−92, 97/−92, 97/−92, 97). The promoters are CMV, RSV, EF-1a, UbC, respectively.
pQBI/Ad/BN/CIITA/Ii-RGC(−92, 97/32, 136/314, 459). The promoters are CMV, RSV, EF-1a, UbC, respectively.
pQBI/Ad/BN/IFN-γ/Ii-RGC(−92, 97/−92, 97/−92, 97). The promoters are CMV, RSV, EF-1a, UbC, respectively.
pQBI/Ad/BN/IFN-γ/Ii-RGC(−92, 97/32, 136/314, 459). The promoters are CMV, RSV, EF-1a, UbC, respectively.

Promoter abbreviations: RSV (rouse sarcoma virus promoter), EF-1a:(human elongation factor-a promoter), UbC (ubiquitin C promoter), CMV (cytomegalovirus promoter).

### Example 9

Construction of Plasmids Contain the Human Ii-RGCS.

Inhibition of human Ii expression by human Ii-RGCs (hIi-RGC) derived from the human Ii gene sequence is disclosed herein. The results of the experiments using hIi-RGCs to inhibit Ii expression in human cells is shown in Table 1. The human Ii cDNA sequence (Strubin et al., EMBO J. 3: 869-72 (1984)) was a gift of Dr. Eric Long. Different lengths of fragments of the Ii gene were generated by PCR using appropriate oligonucleotides. All Ii fragments contain multiple AUG start and stop codons. All were designed to avoid an AUG followed immediately by a stop codon in any reading frame to increase the half life of the antisense RNA. To do this, an AUG was created to override a stop codon in different reading frames. These Ii PCR fragments were cloned into the pcDNA3(+) expression vector by appropriate restriction sites. The human lymphoma cell line, Raji, was used to determine Ii inhibition using these hIi-RGCs. Raji cells were transiently transfected with Polyfect transfection reagent (Qiagen) using 1 μg of hIi-RGC plasmid DNA according to the manufacturer's instructions. After 48 hours incubation, the cells were stained for the expression of Ii and MHC class II and Ii by staining the cells with anti human Ii antibody, LN2 (Pharmingen) and anti-DR antibody (Pharmingen) followed by flowcytometry.

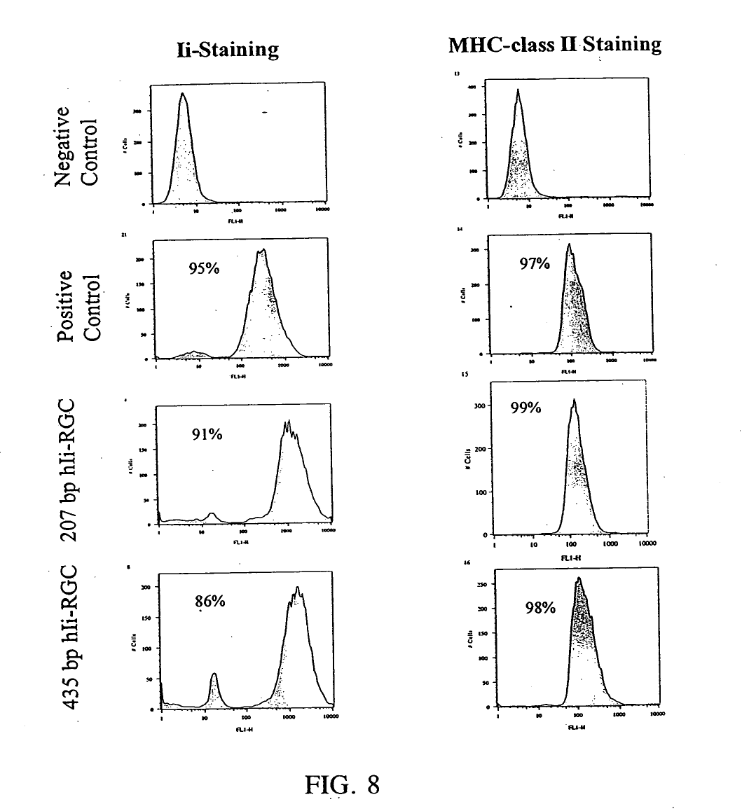

It was observed that Ii-expression was inhibited in a portion of cells (4-9% of cells above the background (see FIG. 8). This inhibition was highly reproducible. In addition, in such a transient transfection assay, it is typical that only 10% of the cells in culture, or fewer, actually take up the added DNA construct. Thus, the 4-9% of cells above background reflects the actual transfection efficiency of the assay system. In these cells, there was no observable affect on MHC class II molecule expression.

TABLE 1
The hli-RGCs tested in human lymphoma line, Raji. + and ++
indicate certain percentage of cells showed profound li suppression
(>95) without the effect of MHC class II molecules.

| Sequence ID # | li inhibition |
|---|---|
| 4 | + |
| 5 | +/− |
| 6 | ++ |
| 7 | − |
| 8 | − |
| 9 | − |
| 10 | − |

In an attempt to maximize the activity of hIi-RGCs, multiple-copy hIi-RGCs (several copies of hIi-RGC in one plasmid) have been made, in which each of expression cassettes is driven by different promoter. These plasmids are listed below.
pQBI/Ad/BN/hIi-RGC(−10, 425/−10, 425). The promoters are CMV, RSV, respectively.
pQBI/Ad/BN/hIi-RGC(−10, 425/−10, 425/−10, 425). The promoters are CMV, RSV, EF-1a, respectively.
pQBI/Ad/BN/CIITA/hIi-RGC(−10, 425/−10, 425). The promoters are UbC, CMV, RSV, respectively.
pQBI/Ad/BN/CIITA/hIi-RGC(−10, 425/−10, 425/−10, 425). The promoters are UbC, CMV, RSV, EF-1a, respectively.
pQBI/Ad/BN/IFN-γ/hIi-RGC(−10, 425/−10, 425/). The promoters are UbC, CMV, RSV, respectively.
pQBI/Ad/BN/IFN-γ/hIi-RGC(−10, 425/−10, 425/−10, 425). The promoters are UbC, CMV, RSV, EF-1a, respectively.

### Example 10

Intratumor Injection of the Ii-RGC Vector Together with IL-2 for Induction of the MHC Class II+/Ii− Phenotype and Therapeutic Efficacy.

BALB/c mice were injected s.c. with 10⁵ Renca cells. At a CIITA:Ii-RGC DNA ratio of 1:6 (w:w), plasmids containing CIITA cDNA gene, CIITA cDNA gene and Ii-RGC(−92, 97), or CIITA gene plus plasmid containing triple Ii-RGC(−92, 97/32, 136/314, 459) were injected into Renca tumors, 0.05-0.2 cm³ in size. 25 μg of total DNA was incubated with DMRIE/C (1,2-dimeristyloxypropyl-3-dimethyl-hydroxy ethyl ammonium bromide/cholesterol) (GIBCO) at a ratio of 1:1 (w/w) one to five minutes before injection. Five days after DNA injection, slides were made from frozen sections of excised tumor. Slides were stained with antibodies against murine MHC Class II and Ii to determine the Class II+/Ii− phenotype of the tumor cells. Staining was also performed with antibodies against CD4, CD8, CD3, CD19 (for B cell) and MAC (macrophage) in order to rule out the possibility that Class II+ cells in the tumor may represent T cells, B cells or macrophages. Results (data not shown) indicated comparable MHC Class II and Ii staining in tumors injected with CIITA alone, while there was evidence of Ii suppression in tumors injected with either CIITA/Ii-RGC (−92, 97) or CIITA plasmid plus plasmid containing triple Ii-RGC. CD4, CD8, and CD3 staining showed very few positive cells, indicating that the Class II+ cells in the tumor were not infiltrating T cells. B cell and macrophage staining also ruled out that the Class II+ cells were not B cells or macrophages. At the same time, slides of spleen samples were also stained with all of the above antibodies as positive controls.

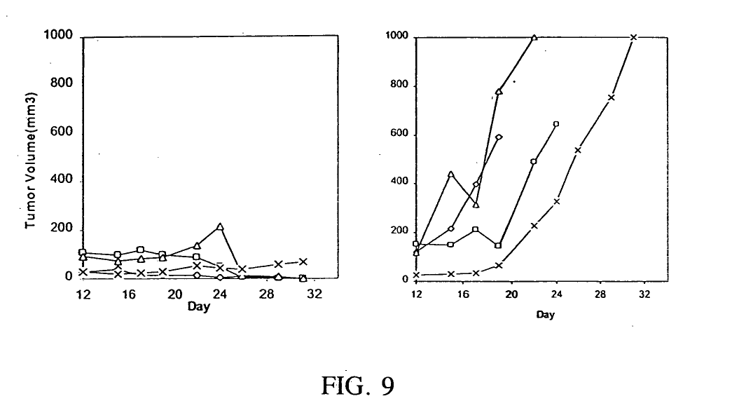

For studies examining the therapeutic efficacy of Ii suppression, BALB/c mice were injected s.c. with Renca renal adenocarcinoma cells and treated by intratumor injection of different plasmid preparations comprising of IL-2 (2 μg), CIITA (3 μg) and Ii-RGC(−92, 97) (18 μg) for day 1 and same preparation without CIITA for days 2-4. Control mice received an empty vector for four consecutive days together with 2 μg IL-2. The tumors were then measured every two to three days. Mice were followed for 31 days and terminated when tumor sizes reached 1000 mm³. The results show that mice treated with CIITA and Ii-RGC containing vectors together with IL-2 exhibited a dramatic reduction in tumor growth, while tumor growth in mice receiving only IL-2 and control vector was progressive and required termination of the mice (see FIG. 9).

### Example 11

### Inhibition of Ii in Human Cells with siRNA Plasmids

SiRNA constructs can reasonably be expected to be as effective as are Ii reverse gene constructs in suppressing Ii protein expression. Here examples of such constructs are revealed to suppress Ii protein expression induced by co-transfection with the Ii cDNA gene. Ten siRNA constructs were tested for inhibition of Ii expression in human kidney line 293 cells. Expressible siRNA constructs, might be preferred to synthetic oligonucleotides for the following reasons. 1) Transfection of cells with RNA oligonucleotides can be more difficult than is transfection with DNA expression constructs. 2) Large scale synthesis of synthetic siRNA oligonucleotides is more expensive than is preparation of a DNA plasmid or other vector. 3) Expression of the construct (and hence the Ii suppressive activity) can be targeted to specific organs or tissues using tissue-specific promoters. 4) The activities of siRNA (whether synthetic or expressed from a genetic vector) is generally much higher than is the activity of reverse gene constructs. For these reasons, expressible siRNA constructs have greater potential benefit for in vivo use.

Design of siRNA(Ii) constructs: Ten siRNA(Ii) constructs were designed, with the oligonucleotides used in their construction presented in Table 2. The constructs were made with the pSuppressorAdeno plasmid (Imgenex. San Diego, Calif.), which was designed specifically for cloning of siRNAs. The plasmid contains both U6 and SV40 promoters optimized for siRNA expression, provides a convenient cloning site for inserting siRNA sequences, and permits delivery to a wide variety of cells. Further, this plasmid can be used also toward construction of a recombinant adenovirus containing the siRNA-expressing construct. Two approaches were followed in the design of these siRNA(Ii) constructs. First, the Imgenex computer program was used to predict 5 constructs (11-15 in Table 2). This program identifies RNA sequences that have a base composition likely to hybridize to the Ii RNA (i.e., appropriate G-C content, etc.). The resulting 5 siRNA(Ii) constructs (11-15 in Table 2) are expected to be potent inhibitors if they actually hybridize with Ii mRNA. However, since the tertiary structure of any given mRNA is difficult to predict, such computer-designed siRNA(Ii) constructs might not be found experimentally to be accessible to Ii mRNA. Therefore, a second approach was also used in the design of 5 additional constructs (16-20 in Table 2). Previous data on the use of Ii-RGC to inhibit expression of Ii protein revealed that some Ii antisense oligonucleotides (Qiu Cancer Imm Immunother. 48:499-506 (1999) (Xu U.S. Pat. No. 6,368,855) and Ii-reverse gene constructs (RGC; Lu Cancer Immunol Immunother. 52: 592-598 (2003)) (U.S. application Ser. No. 10/127,347), hybridize to the first 400 bp of human Ii mRNA, with potent consequent inhibition of Ii protein expression. One can deduce from these data, that this region of human Ii mRNA should be largely accessible to siRNA constructs. This proposal, furthermore, is consistent with the data in the literature that the mRNA region containing the AUG site starting translation is generally a sensitive region for antisense constructs to bind to mRNA. Therefore, by inspection, another 5 μl siRNA constructs were designed to hybridize to sections of Ii mRNA within the first 400 bp of human Ii mRNA around the AUG start site. Because there are two AUGs at the beginning of the human Ii mRNA, both of which appear to be functional translation start sites, siRNA sequences were designed to target both of these sites. Specifically, two overlapping sequences were designed around the first AUG and three overlapping siRNA sequences were designed around the second AUG. While these 5 siRNA (Ii) sequences might not have optimal annealing parameters, they can be expected to hybridize with Ii RNA. All sequences were designed with a short loop sequence to allow for hairpin formation of the expressed siRNA sequences. The formation of a hairpin results in a functional double-stranded siRNA. The requirement for double-stranded RNA in forming the RNA-induced silencing complex (RISC) that interacts with and cleaves target mRNA, has been clearly demonstrated (Nature Reviews Genetics 2:110-119, 2001).

TABLE 2
Structure of 10 SiRNA constructs.

| SEQ | | |
|---|---|---|
| ID NO. | Position | sequences |
| 11 | 1-21 | 5′tcgattcccagatgcacaggaggagatcgat |
| | | ctcctcctgtgcatctgggaattttt |
| 12 | 8-28 | 5′tcgaatgcacaggaggagaagcaggatcgat |
| | | cctgcttctcctcctgtgcatttttt |
| 13 | 47-67 | 5′tcgaaagccagtcatggatgaccagatcgat |
| | | ctggtcatccatgactggcttttttt |
| 14 | 56-76 | 5′tcgaatggatgaccagcgcgaccttatcgat |
| | | aaggtcgcgctggtcatccatttttt |
| 15 | 84-104 | 5′tcgacaatgagcaactgcccatgctatcgat |
| | | agcatgggcagttgctcattgttttt |
| 16 | 267-287 | 5′tcgacctgcagctggagaacctgcgatcgat |
| | | cgcaggttctccagctgcaggttttt |
| 17 | 312-332 | 5′tcgagcctgtgagcaagatgcgcatatcgat |
| | | atgcgcatcttgctcacaggcttttt |
| 18 | 396-416 | 5′tcgatgccaccaagtatggcaacatatcgat |
| | | atgttgccatacttggtggcattttt |
| 19 | 414-434 | 5′tcgacatgacagaggaccatgtgatatcgat |
| | | atcacatggtcctctgtcatgttttt |
| 20 | 501-521 | 5′tcgacctgagacaccttaagaacacatcgat |
| | | gtgttcttaaggtgtctcaggttttt |

A double-stranded oligonucleotide is created by annealing two oligonucleotides coding for shRNA (short hairpin RNA) respectively for sense and complementary strands as indicated above. The annealed oligonucleotides will have “tcga” (shown above) and “gatc” overhangs to assist cloning into the Sal I and Xba I digested pSuppressor vectors. The sense sequence is single-underlined. The loop sequence is bold. The inverted sequence is double-underlined.

Experimental Procedures and Results. 10 siRNA(Ii) constructs were created by cloning the above sequences into pSuppressorAdeno plasmid (Imgenex, San Diego, Calif.) using Sal1 and Xba1 enzyme sites, according to standard molecular biological techniques. Cells of the 293 human kidney line (ATCC Number CRL-1573) were co-transfected with human Ii cDNA gene plasmid (0.18 μg) with each of these Ii siRNA constructs (0.82 μg). Several active Ii siRNA construct(s) were defined. Briefly, 293 cells (2×10⁵/well) were cultured in 6-well plates overnight. DNA mixtures were transfected into 293 cells using Effectene transfection reagent (Qiagen, Valencia, Calif.) according to the manufacturer's instruction. Cells were incubated in a CO₂ incubator at 37° C. for 36 hours. Cells were intracellular stained with anti-human Ii antibody (LN-2, Pharmingen, San Diego, Calif.) and then analyzed by flow-cytometry (Table 3). The sensitivity (gating) of the instrument was set such that 99% of Ii-negative 293 cells could be detected. Ii cDNA was co-transfected with empty pSuppressorAdeno plasmid as a positive control, i.e., without Ii suppression. For each of three separate experiments, the percentages of Ii+ cells were determined for cells transfected with empty pSuppressorAdeno plasmid or each of the respective ten siRNA plasmids (Table 3). The difference from Ii+control cells in each case reflects the degree of Ii suppression by the various siRNA constructs. The mean suppression (29%) of plasmids 11-18 is substantial. From these data one can conclude that plasmids 11-18 (mean suppression of 29%) have potent activity and plasmids 19, 20, and “empty” have no Ii-suppressing activity.

TABLE 3
li suppression by siRNA constructs.

| | Experi 1 | | | Experi 1 | | | Experi 1 | | | Mean |
|---|---|---|---|---|---|---|---|---|---|---|
| Plamid | Obs | Diff | % sup | Obs | Diff | % sup | Obs | Diff | % sup | sup |
| Empty | 47.3 | | | 60.0 | | | 49.9 | | | |
| 11 | 39.6 | −7.7 | 16 | 53.8 | −6.2 | 10 | 32.7 | −27.3 | 55 | 27 |
| 12 | 31.1 | −16.2 | 34 | 50.9 | −9.1 | 15 | 39.3 | −20.7 | 41 | 30 |
| 13 | 28.7 | −18.6 | 39 | 59.0 | −1.0 | 2 | 39.9 | −20.1 | 40 | 27 |
| 14 | 18.6 | −28.7 | 61 | 49.4 | −10.6 | 18 | 38.2 | −21.8 | 44 | 41 |
| 15 | 33.3 | −14.0 | 30 | 52.3 | −7.7 | 13 | 45.8 | −14.2 | 28 | 24 |
| 16 | 30.2 | −17.1 | 36 | 47.9 | −12.1 | 20 | 35.8 | −24.2 | 48 | 35 |
| 17 | 42.0 | −5.3 | 11 | 44.4 | −15.6 | 26 | 39.8 | −20.2 | 40 | 26 |
| 18 | 33.7 | −13.6 | 29 | 52.5 | −7.5 | 13 | 51.6 | −8.4 | 17 | 19 |
| 19 | 49.1 | 1.8 | −4 | 55.5 | −4.5 | 8 | | | | 2 |
| 20 | 40.6 | −6.7 | 14 | 63.2 | 3.2 | 5 | | | | 4 |

The structures of the respective plasmids 11-20 are indicated in Table 3. Obs=observed percentage of Ii+ cells. Diff=difference in observed % from percentage found with “empty” plasmid. % sup=percentage suppression (difference/observed percentage with “empty” plasmid. Mean sup=mean of the % suppression over three experiments.

Experimental methods for testing effects of siRNA in Raji cells. The gene gun delivery method was used to transfect the siRNA constructs into Raji cells. Plasmid DNA was precipitated onto gold particles. Gold microcarriers (0.5 mg of 1 μm particles) were suspended by sonication in 100 μl of 0.05 M spermidine. The indicated amount of DNA at a concentration of 1 mg/ml in endotoxin-free water was added and sonicated and 100 μl of 1 M CaCl₂ was added dropwise. This gold-DNA mixture was allowed to stand for 10 min before being washed 3 times with 250 μl of 100% ethanol. After the final wash, the pellet was resuspended in 200 μl of 0.025 mg/ml polyvinylpyrrolidone (PVP) in 100% ethanol, transferred to a 15 ml tube, and made up to 1 ml with PVP/ethanol. The resulting microcarrier loading quantity (MLQ) of 0.5 mg of gold per shot and a variable DNA loading ratio (DLR) was delivered to mice. One ml of DNA/microcarrier suspension produced 17 coated 0.5-inch cartridges, which were stored overnight at 4° C. with desiccant prior to use. For vaccinating mice by the gene gun delivery method, the fur of the abdomen of each mouse was be removed with electric clippers prior to each vaccination. The barrel of the gene gun was held directly against the abdominal skin, and a single microcarrier shot was delivered using a helium pressure of 400-500 psi. Injections were performed using a helium-activated Gene Gun System (PowderJect).

### Example 12

### Inhibition of Ii in Human Cells by siRNA(Ii) Duplexes

In addition to the use of shRNAs to silence Ii gene expression a second method was tested that involved chemical synthesis of siRNA duplexes. Synthetic siRNAs offer some advantages over siRNA plasmid vectors. First, the delivery of synthetic siRNAs does not involve the introduction of foreign plasmid DNA, which can have deleterious effects on eukaryotic cells including insertional mutagenesis. Second, synthetic siRNAs result in transient gene suppression which may be more efficacious for therapeutic purposes, such as presented herein.

Design and testing of double-stranded siRNA. Antisense RNA is capable of silencing specific genes when introduced into cells (Guo, Cell 81, 611). Using C. elegans it was demonstrated that injection of double-stranded RNA was more effective in gene silencing than injection of sense or antisense strands alone (Fire, Nature 391, 806). Therefore additional siRNA(Ii) were designed and synthesized by Qiagen (Valencia, Calif.) (Tables 4 and 5). Rational siRNA design and stringent homology analysis are critical for achieving optimal silencing of target genes and for minimizing off-target effects. QIAGEN has licensed the HiPerformance design algorithm from Novartis Pharmaceuticals for the selection of highly functional target sequences for RNAi. The algorithm is based on the largest independent study of siRNA functionality to date, in which the gene silencing efficiency of more than 3000 synthetic siRNA duplexes directed against 34 targets was analyzed. These data were used to develop a sophisticated pattern recognition algorithm. The HiPerformance design algorithm is integrated with a proprietary homology analysis tool and a comprehensive non-redundant gene database, to allow thorough and accurate homology analysis. As a result, custom-designed 4-for-Silencing siRNA Duplexes provide highly specific and potent siRNA. 4-for-Silencing siRNA Duplexes are highly pure HPP Grade siRNA. High purity increases siRNA specificity and reduces the possibility of off-target effects.

Experimental Results. The following experiment revealed that siRNAs specific for the invariant chain (Ii) which is associated with MHC class II molecules inhibit expression of Ii in siRNA-transfected human cells. For these experiments, HeLa cells were plated at 2.5×10⁴ cells per well in 6-well plates 24 hours before transfection. HeLa cells were transfected with 4 siRNA(Ii)s specific for the invariant chain (Ii) using the siRNAfect transfection reagent (Qiagen Inc.) according to the manufacturer's recommendations. siRNAs specific for Lamin A/C and a non-silencing fluorescein-labeled siRNA were used as controls for Ii gene silencing. Cells were treated with interferon gamma (IFN-γ) 100 units/ml and thyroxine 1×10⁻⁷ M to induce MHC class II expression 6 hours after transfection. 48 hrs post-transfection cells were stained with antibodies to Ii, HLA-DR and isotype control. The cells were FACS-analyzed as in Example 11 (Table 6). All four of the siRNA(Ii) duplexes revealed significant suppression of Ii protein expression.

TABLE 4
Design of 4 siRNA(Ii)s and their positions in Ii RNA sequence

BASE COUNT 326 a 431 c 327 g 243 t
ORIGIN

| | |
|---|---|
| 1 | cagggtccca gatgcacagg aggagaagca ggagctgtcg ggaagatcag aagccagtca |
| 61 | tggatgacca gcgcgacctt atctccaaca atgagcaact gcccatgctg ggccggcgcc |
| 121 | ctggggcccc ggagagcaag tgcagccgcg gagccctgta cacaggcttt tccatcctgg |
| 181 | tgactctgct cctcgctggc caggccacca ccgcctactt cctgtaccag cagcagggcc |
| 241 | ggctggacaa actgacagtc acctcccaga acctgcagct ggagaacctg cgcatgaagc |
| 301 | ttcccaagcc tcccaagcct gtgagcaaga tgcgcatggc caccccgctg ctgatgcagg |
| 361 | cgctgcccat gggagccctg ccccaggggc ccatgcagaa tgccaccaag tatggcaaca |
| 421 | tgacagagga ccatgtgatg cacctgctcc agaatgctga ccccctgaag gtgtacccgc |
| 481 | cactgaaggg gagcttcccg gagaacctga gacaccttaa gaacaccatg gagaccatag |
| 541 | actggaaggt ctttgagagc tggatgcacc attggctcct gtttgaaatg agcaggcact |
| 601 | ccttggagca aaagcccact gacgctccac cgaaagagtc actggaactg gaggacccgt |
| 661 | cttctgggct gggtgtgacc aagcaggatc tgggcccagt ccccatgtga gagcagcaga |
| 721 | ggcggtcttc aacatcctgc cagccccaca cagctacagc tttcttgctc ccttcagccc |
| 781 | ccagcccctc ccccatctcc caccctgtac ctcatcccat gagaccctgg tgcctggctc |
| 841 | tttcgtcacc cttggacaag acaaaccaag tcggaacagc agataacaat gcagcaaggc |
| 901 | cctgctgccc aatctccatc tgtcaacagg ggcgtgaggt cccaggaagt ggccaaaagc |
| 961 | tagacagatc cccgttcctg acatcacagc agcctccaac acaaggctcc aagacctagg |
| 1021 | ctcatggacg agatgggaag gcacagggag aagggataac cctacaccca gaccccaggc |
| 1081 | tggacatgct gactgtcctc tcccctccag cctttggcct tggcttttct agcctattta |
| 1141 | cctgcaggct gagccactct cttccctttc cccagcatca ctccccaagg aagagccaat |
| 1201 | gttttccacc cataatcctt tctgccgacc cctagttccc tctgctcagc caagcttgtt |
| 1261 | atcagctttc agggccatgg ttcacattag aataaaaggt agtaattaga aaaaaaaaaa |
| 1321 | aaaaaaa |

TABLE 5

Sequences of synthesized siRNA
duplexes with terminal overhangs.

| | | |
|---|---|---|
| (I) siRNA(Ii) | 5′ CCAUUGGCUCCUGUU | (SEQ ID NO: 21) |
| | UGAAUU 3′ | |
| | 3′ UUCAAACAGGAGC | (SEQ ID NO: 22) |
| | CAAUGGUG 5′ | |
| (II) siRNA(Ii) | 5′ CACUGACGCUCCACC | (SEQ ID NO: 23) |
| | GAAAUU 3′ | |
| | 3′ UUUCGGUGGAGCGUC | (SEQ ID NO: 24) |
| | AGUGGG 5′ | |
| (III) siRNA(Ii) | 5′ GAACUGGAGGACCCG | (SEQ ID NO: 25) |
| | UCUUUU 3′ | |
| | 3′ AAGACGGGUCCUCCA | (SEQ ID NO: 26) |
| | GUUCCA 5′ | |
| (IV) siRNA(Ii) | 5′ GGGUGUGACCAAGCA | (SEQ ID NO: 27) |
| | GGAUUU 3′ | |
| | 3′ AUCCUGCUUGGUCAC | (SEQ ID NO: 28) |
| | ACCCAG 5′ | |

TABLE 6
li suppression by siRNA.

| siRNA transfection | % li-positive cells | Mean suppression |
|---|---|---|
| HeLa unstained | 0.0 | |
| HeLa (untreated) li antibody | 5.6 | |
| HeLa li-expressing + control | 89.1 | |
| HeLa li-expressing + lamin siRNA | 89.0 | |
| HeLa li-expressing + siRNA(li)-I | 39.3 | 49.8 |
| HeLa li-expressing + siRNA(li)-II | 36.6 | 52.5 |
| HeLa li-expressing + siRNA(li)-III | 52.6 | 36.5 |
| HeLa li-expressing + siRNA(li)-IV | 40.2 | 48.9 |

### Example 13

### Immunomodulation of HIV gp120 DNA Vaccine by Ii Suppression

Induction of Ii protein suppression in either dendritic cells or other professional antigen presenting cells, into which a DNA coding for a vaccine antigen also has been introduced, leads to a potent T helper cell response which enhances both T helper cell memory and cytotoxic T cell (CTL) responses. Such a response enables a potent therapeutic effect of DNA vaccines, which has previously been lacking.

The mechanism for this effect, as presented in detail in the Background of this Disclosure depends upon suppression of Ii protein expression in the endoplasmic reticulum (ER) of the antigen-presenting cell (APC). Cytoplasmic peptides which are processed by proteosomes and transported into the ER for binding there to MHC class II molecules; can also become bound to major histocompatibility complex (MHC) class II molecules which are not blocked by the Ii protein. Normally, the Ii protein blocks the antigenic peptide binding site of MHC class II molecules until the trimer (MHC class IIα and β chains+Ii protein) are transported to a post-Golgi compartment into which selected external antigen has been transported for proteolytic digestion of the antigen, along with proteolysis of the Ii protein, and binding of the antigenic peptides into the MHC class II molecules.

The altered, ER binding site of MHC class II molecules in cells with suppressed expression of the Ii protein expands the repertoire of MHC class II epitopes which are bound into MHC class molecules, which continue their path of intracellular transport to the cell surface for presentation to T helper cells. Additionally, the potency of presentation of many epitopes is increased because a large fraction of the MHC class II molecules come to bind and express determinants synthesized from the transfected DNA vaccine gene. The dose of that gene, potency of it's promoter, effect of stability of the cytoplasmically synthesized protein or protein fragment upon proteosomes processing, and other factors, all contribute to the concentration in the ER of vaccine peptides which can become bound to the “unblocked” MHC class II molecules in the ER.

As revealed in this example, the response to immunizing mice with a DNA for HIV gp120 antigen is greatly enhanced by co-immunizing mice with Ii Reverse Gene Construct (Ii-RGC), which induces transcription of a RNA which hybridizes with the mRNA for the Ii protein, leading to inhibition of Ii protein expression. An advantage of the gold bead immunization technology is that the final, optimally effective ratios and concentrations of plasmid DNA for the DNA vaccine and for the Ii-RGC can be administered of a per cell basis, within the cells into which the gold bead-absorbed DNAs are impelled.

Experiment 1

Preparation of DNA-Coated Gold Beads. Prior to coating the gold beads with DNA, the following parameters should be determined for each study: gold bead load ratio per cartridge (GLR), DNA load ratio per cartridge (DLR), DNA/gold beads ratio (DGR), the number of cartridges (shots) to be used per immunization, and the number of immunizations needed. In following experiment 1 and 2, DGR=4, GLR=0.5, DLR=2.

TABLE 7
Calculation of DNA and Gold Beads

| | | | | | Gold |
|---|---|---|---|---|---|
| | | | | | beads |
| DGR | GLR | DLR | No. of | DNA needed | needed |
| μg/mg | mg/cartridge | μg/cartridge | cartridges | (μg) | (mg) |
| 4 | 0.5 | 2 | 60 | 120 | 30 |
| 2 | 0.5 | 1 | 60 | 60 | 30 |
| 1 | 0.5 | 0.5 | 60 | 30 | 30 |
| 0.5 | 0.5 | 0.25 | 60 | 15 | 30 |
| 4 | 1 | 4 | 60 | 240 | 60 |
| 2 | 1 | 2 | 60 | 120 | 60 |
| 1 | 1 | 1 | 60 | 60 | 60 |
| 0.5 | 1 | 0.5 | 60 | 30 | 60 |

Immunization of mice. Female BALB/c mice (6-8 week old) are anesthetized with a solution comprising ketamine solution (100 mg/mL) 200 μL, xylazine solution (20 mg/mL) 250 μL, and normal saline 300 μL (total 750 μL), each mouse receive i.p. injections of 50 μL at 6 weeks. And then mice are shaved promptly with an electric shaver and subjected to the gene gun shooting. The gun will be 0.0 to 0.5 cm from the skin of a mouse. Shooting with 400 psi helium gas. Each mouse was given 4 shots, without later boosts. Three weeks later, in vitro did IFN-γ Elispot assay use long P18 peptide(RIQRGPGRAFVTIGK) and short P18 peptide (RGPGRAFVTI), respectively.

ELISPOT Assay. ELISPOT assays were performed according to the commercially available protocols of Cellular Limited Technology. Briefly, 100 μl of a solution of the cytokine-specific capture antibody at 6 μg/mL in 0.01 M sodium phosphate, 0.14 M sodium chloride, pH 7.2 (phosphate-buffered saline solution, PBS), is added to each well of a 96-well Immunospot plate (M200) for an overnight incubation at 4° C. After aspiration, 200 μl of phosphate-buffered saline solution containing 10% fetal bovine serum and 1% penicillin-streptomycin-glutamine is added to each well for 2 hr at RT. After washing four times with 1% Tween-20 in PBS, 100 μl of single cell suspensions from the spleens of immunized mice, at 10⁵ to 10⁶ cells/well are re-stimulated with 100 μl of epitope only peptide at 5 μg/well or 25 μg/well in medium and incubated for 24-72 hr at 37° C., 5% CO₂. After washing two times with PBS and four times with wash buffer 1,100 μl of 2 μg/ml biotinylated anti-human IFN-γ in PBS with 10% fetal bovine serum (dilution buffer) is added to each well for 2 hr at RT. After washing five times with wash buffer 1,100 μl of streptavidin-horse radish peroxidase conjugate in dilution buffer is added to each well for 1 hr at RT. After washing four times with wash buffer I and twice with PBS, 100 μl of the 3-amino-9-ethylcarbazole/H₂O₂ substrate (Pharmingen 551951) is added for 30-60 min in the dark at RT. The reaction is stopped by washing three times with 200 μl of de-ionized water. ELISPOT data analysis is performed by using the Immunospot 1.7e software (Cellular Limited Technology).

TABLE 8
Enhancement of HIV gp120 DNA vaccine response

| | | | | | | No. of IFN-γ Spots | | |
|---|---|---|---|---|---|---|---|---|
| Group/ | | GMCSF | CIITA | li-RGC | pBud.CE4 | Long | Short | Medium |
| mice | Antigen | (μg) | (ng) | (μg) | (μg) | p18 | p18 | only |
| 1/3 | 0 | 0 | 0 | 0 | 0 | 3 | 4 | 3 |
| 2/3 | *Rsv/gp120 | 0 | 0 | 0 | 0.8 | 10 | 9 | 2 |
| | (1.2 μg) | | | | | | | |
| 3/3 | RSV/gp120 | 0.25 | 0 | 0 | 0.55 | 50 | 48 | 4 |
| | (1.2 μg) | | | | | | | |
| 4/3 | RSV/gp120 | 0.25 | 50 | 0 | 0.5 | 15 | 13 | 3 |
| | (1.2 μg) | | | | | | | |
| 5/3 | RSV/gp120 | 0.25 | 50 | 0.5 | 0 | 200 | 190 | 8 |
| | (1.2 μg) | | | | | | | |
| 6/3 | **Ad/BN/gp120 | 0.25 | 50 | 0 | 0.5 | 3 | 5 | 2 |
| | (1.2 μg) | | | | | | | |
| 7/3 | Ad/BN/gp120 | 0.25 | 50 | 0.5 | 0 | 4 | 5 | 3 |
| | (1.2 μg) | | | | | | | |

*In construct RSV/gp120, gp120 gene has leader sequence
**In Ad/BN/gp120, gp120 gene no leader sequence.

Experiment 2

Experimental procedures are the same as for Experiment 1.

TABLE 9
Enhancement of HIV gp120 DNA vaccine response

| | | | | | | No. of IFN-γ Spots | | |
|---|---|---|---|---|---|---|---|---|
| Group/ | | GMCSF | CIITA | li-RGC | pBud.CE4 | Long | Short | Medium |
| mice | Antigen | (μg) | (ng) | (μg) | (μg) | p18 | p18 | only |
| 1/3 | 0 | 0 | 0 | 0 | 0 | 8 | 6 | 6 |
| 2/3 | RSV/gp120 | 0.25 | 0 | 0.5 | 0.05 | 300 | 290 | 10 |
| | (1.2μg) | | | | | | | |

As revealed in the above data, co-injection of the DNAs for HIV gp120 plus Ii-RGC leads to substantial enhancement of the CD4+ T cell IFN-γ responses, both in terms of cell number and output per cell (spot size). This enhanced response will lead to more potent CTL and a stronger CD4+ memory cell response.

The above experiments also demonstrate that co-injection of the MHC class II transactivator (CIITA) induces a suppressed response relative to that seen without CIITA. This pattern results from the fact that DNA-coated beads impelled into the keratinocytes result in two patterns of response, depending upon whether CIITA DNA is also on the beads. Without CIITA the vaccine DNA leads to expression of HIV gp120, expression of MHC class I determinants, which can prime a MHC class I-restricted CTL response. Otherwise, HIV gp120 antigen might be released by keratinocytes and then scavenged by macrophages or dendritic cells for presentation by MHC class II molecules of those cells. The volume of T cell-presented MHC class II epitopes from protein synthesized from a DNA impelled into keratinocytes is very low, compared at least to the volume of MHC class I epitopes presented by such cells. In such cells the expressed Ii RGC has no useful function since these cells do not express either MHC class II molecules or the Ii protein. The endogenously synthesized antigen protein does not become expressed except rarely after either vesicular transport to the post-Golgi antigen charging component, except after extracellular release and uptake by professional APC.

However, when the keratinocytes are concurrently transfected with CIITA, MHC class II molecules are expressed on keratinocytes. If Ii-RGC is not co-transfected into such cells the expression of MHC class II molecules is of no consequence for MHC class II epitope presentation by those cells because they lack the remainder of the MHC class II processing and presentation functions which are present in the post-Golgi antigen charging component of professional APCs. But, when Ii suppression also occurs due to Ii RGC constructs, siRNA(Ii)s or antisense Ii oligonucleotides, then MHC class II epitopes can be presented by the gene-transfected keratinocytes. However, the biological type of the response in such cells is now a suppression phenotype rather than an activating phenotype. That occurs because presentation of epitopes to T helper cells by MHC class II molecules in the absence of B7.1, B7.2, CD40, CD80, CD86, and other APC cofactors for T cell presentation. The default response pathway for T cell activation by antigenic epitopes presented by MHC class II molecules without cofactors is a Th2 suppressing phenotype. Methods for the induction and use of such immunosuppressing effects are incorporated by reference from U.S. Pat. No. 6,106,840, U.S. Pat. No. 6,218,132 and U.S. Pat. No. 6,405,796. Autoimmune diseases can be treated by this method of suppression induction when at least one principal antigenic antigen associated with the pathogenesis of the disease is known and a DNA coding part, or all of the antigen is available. A treatment protocol consists of administering, for example on gold beads at defined concentrations and ratios the following three DNAs: the DNA for the pathogenesis-associated antigen, the DNA for an Ii-RGC plasmid and the DNA for CIITA. Such immunizations are performed in the dose, schedule and methods and with such adjuvant which are specified in the following U.S. Pat. No. 6,710,035, U.S. Pat. No. 6,586,409, U.S. Pat. No. 6,214,804, U.S. Pat. No. 6,339,068, U.S. Pat. No. 5,620,896, U.S. Pat. Nos. 6,706,694, 6,649,409, U.S. Pat. No. 6,258,799, U.S. Pat. No. 6,743,444, U.S. Pat. No. 6,656,706, and U.S. Pat. No. 6,783,759.

In one aspect this method of therapy can be applied to the treatment of autoimmune diseases such as rheumatoid arthritis, multiple sclerosis, and diabetes mellitus-type I. For example, DNA vaccines for human myelin basic protein, oligogliodendrocyte protein, and other MS-related antigens can be administered with both CIITA and Ii-RGC to suppress multiple sclerosis. Likewise, DNAs for hcgp42, collagen, and rheumatoid arthritis- or osteoarthritis-related antigens can be administered with both CIITA and Ii-RGC to suppress rheumatoid arthritis. DNAs for insulin, glutamic acid decarboxylase, glucose transporter-2 and other Type I diabetes mellitus-related antigens can be administered with both CITTA and Ii-RGC to suppress type I diabetes mellitus. In another aspect, graft rejection can be treated by this method to suppress rejection when a suitable antigen of the grafted tissue with an encoding cDNA is known. A treatment protocol consists of administering, for example, gold beads with defined concentrations and ratios the following three DNAs: the DNA for the transplant rejection-associated antigen, the DNA for CIITA, and the DNA for Ii-RGC.

### Example 14

### Suppression of Ii in Human Dendritic Cells by Human Ii siRNA Plasmids

SiRNAs were used to suppress expression of Ii protein in fresh human peripheral blood monocyte-derived dendritic cells. Human monocytic dendritic cells were prepared from a human peripheral blood, commercial preparation (Leukopack from All Cells, Inc. Boston). The peripheral blood mononuclear cells (PBMC) (2.5×10⁶) were incubated in 25 ml DC medium comprising X-VIVO 15 culture medium (Cat. No. 04-418, Cambrex Bioscience Walkersville, Inc., Walkersville, Md.), 10% human AB serum (Cat. No. 100-512, Gemini Bio-Prodcuts, Woodland, Calif.), 1% penicillin-streptomycin-glutamine stock solution (Cat. No. 10378-016, GIBCO, Grand Isle N.Y.; with stock solution concentrations: penicillin 10,000 U/ml; streptomycin 10,000 ug/ml; L-glutamine, 29.2 mg/ml), overnight at 37° C. in a 5% CO₂ atmosphere to allow the monocytes to attach to the plastic well bottoms. Non-adherent T-cells and B-cells were removed by gentle washing with 0.1 M sodium phosphate-buffered, 0.14 M NaCl solution, pH 7.4 (phosphate-buffered saline; PBS). Adherent monocytes were then incubated in 25 ml DC medium containing 0.2-2 ng/ml IL-4 (R&D Systems, Minneapolis, Minn.), and 0.2-2 ng/ml GM-CSF (R&D Systems) for 7 days at 37° C. in a 5% CO₂ atmosphere to permit the differentiation of the monocytes into dendritic cells. That medium was changed on days 3 and 6. The dendritic cells were collected by trypsinization and resuspended in DC medium with 2 ng/ml IL-4 (R&D Systems), and 0.2-2 ng/ml GM-CSF (R&D Systems). From about 2.5×10⁶ PBMC of the Leukopack, about 2×10⁶ dendritic cells were obtained after 7 days of culture. Those monocyte-derived dendritic cells were plated in 6-well plates at 3.3×10⁵ cells per well in 3 ml DC culture medium with IL-4 and GM-CSF. Polyethyleneimine (PEI; Cat. No 408727, Sigma Aldrich, St. Louis, Mo.) formulations of the plasmids were added to the cultures and incubated at 37° C. in a 5% CO₂ atmosphere to transfect the dendritic cells with the control and human Ii siRNA expressing plasmid DNAs (Table 10). 48 hr post-transfection cells were stained with antibodies to Ii. The cells were FACS-analyzed as in Example 11. The two human Ii siRNA expressing plasmids induced significant suppression of Ii protein expression in human dendritic cells.

TABLE 10
Transfection and FACS analysis of human dendritic cells treated
with PEl-formulated Human li siRNA plasmids

| Treatment | % li Positive Cells | % li Inhibition |
|---|---|---|
| Empty Vector | 62.7 | — |
| P4 | 30.5 | 51.4 |
| P7 | 32.5 | 48.1 |
| P4 + P7 | 26.7 | 57.4 |

1. A composition comprising an siRNA effective to inhibit Ii expression.

2. The composition of claim 1 wherein the siRNA comprises an RNA duplex comprising:
a) a first strand comprising a sense sequence of Ii, the sense sequence of Ii of from 10 to 25 nucleotides in length; and
b) a second strand comprising a reverse complement of the sense sequence in a).

3. The composition of claim 2 wherein the siRNA comprises an RNA duplex comprising:
a) a first strand comprising a sense sequence of Ii, the sense sequence of Ii of from 19 to 25 nucleotides in length; and
b) a second strand comprising a reverse complement of the sense sequence in a).

4. The composition of claim 3 wherein the siRNA comprises an RNA duplex comprising:
a) a first strand comprising a 21 to 23 nucleotide sense sequence of Ii; and
b) a second strand comprising a reverse complement of the sequence in a).

5. A composition comprising a DNA sequence which encodes an siRNA effective to inhibit Ii expression.

6. The composition of claim 5 wherein the DNA sequence is in a plasmid vector.

7. The composition of claim 5 wherein the DNA sequence is in a viral vector.

8. The composition of claim 7 wherein the viral vector is selected from the group consisting of adenovirus, adeno-associated virus, lentivirus, poxvirus, influenza, and retrovirus.

9. The composition of claims 1 or 5 wherein the siRNA comprises in a single molecule:
a) a sense sequence of Ii of from 10 to 25 nucleotides in length;
b) a reverse complement of the sequence in a); and
c) an intervening sequence enabling duplex formation between the sense and reverse complement sequences.

10. The composition of claim 9 wherein the siRNA comprises in a single molecule:
a) a sense sequence of Ii of from 19 to 25 nucleotides in length;
b) a reverse complement of the sequence in a); and
c) an intervening sequence enabling duplex formation between the sense and reverse complement sequences.

11. The composition of claim 10 wherein the siRNA comprises in a single molecule:
a) a 21 to 23 nucleotide sense sequence of Ii;
b) a reverse complement of the sequence in a); and
c) an intervening sequence enabling duplex formation between the sense and reverse complement sequences.

12. The composition of claims 1 or 5 wherein the siRNA comprises the RNA of a sequence selected from the group consisting of SEQ ID NO: 11, SEQ ID NO: 12, SEQ ID NO: 13, SEQ ID NO: 14, SEQ ID NO: 15, SEQ ID NO: 16, SEQ ID NO: 17, and SEQ ID NO: 18.

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. A method for inhibiting expression of Ii in a cell, the method comprising introducing an siRNA into a cell expressing Ii, wherein the siRNA is introduced either directly or indirectly into the cell, and further wherein the siRNA is capable of forming an RNA-induced silencing complex, thereby inhibiting expression of Ii in the cell.

43. The method of claim 42 wherein the siRNA is introduced indirectly into the cell, the siRNA being transcribed within the cell from an expressible nucleic acid sequence or sequences encoding the siRNA.

44. The method of claim 43 wherein the expressible nucleic acid sequence or sequences comprise:
a) a first expressible DNA sequence which encodes a first RNA sequence comprising a sense sequence of Ii; and
b) a second expressible DNA sequence which encodes a second RNA sequence comprising the reverse complement of the sense sequence of Ii in step a),
wherein an siRNA duplex forms upon hybridization of the first RNA sequence to the second RNA sequence.

45. The method of claim 44 wherein at least one of the first and second expressible DNAs is a purified PCR product.

46. The method of claim 44 wherein at least one of the first and second expressible DNAs is in a vector.

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91. (canceled)

92. (canceled)

93. (canceled)

94. (canceled)

95. (canceled)

96. (canceled)

97. (canceled)

98. (canceled)

99. (canceled)

100. (canceled)

101. (canceled)

102. (canceled)

103. (canceled)

104. (canceled)

105. (canceled)

106. (canceled)

107. (canceled)

108. (canceled)

109. (canceled)

110. (canceled)

111. (canceled)

112. (canceled)

113. (canceled)

114. (canceled)

115. (canceled)

116. (canceled)

117. (canceled)

118. (canceled)

119. (canceled)

120. (canceled)

121. (canceled)

122. (canceled)

123. (canceled)

124. (canceled)

125. (canceled)

126. (canceled)

127. (canceled)

128. (canceled)

129. (canceled)

130. (canceled)

131. (canceled)

132. (canceled)

133. (canceled)

134. (canceled)

135. (canceled)

136. (canceled)

137. (canceled)

138. (canceled)

139. (canceled)

140. (canceled)

141. (canceled)

142. (canceled)

143. (canceled)

144. (canceled)

145. (canceled)

146. (canceled)

147. (canceled)

148. (canceled)

149. (canceled)

150. (canceled)

151. (canceled)

152. (canceled)

153. (canceled)

154. (canceled)

155. (canceled)

156. (canceled)

157. (canceled)

158. (canceled)

159. (canceled)
